(19) United States
(12) Patent Application Publication
(10) Pub. No.: US 2026/0315363 A1
(43) Pub. Date: Oct. 15, 2026

(54) Liquid Emulsion Comprising Ethylcellulose, a Natural Resin, a Liquid Fatty Alcohol, a Solid Ester and/or a Polar Hydrocarbon-Based Oil and Process Using Same

(71) Applicant: L'OREAL, Paris (FR)

(72) Inventors: Florence LAHOUSSE, Chevilly Larue (FR); Régine IMBERT, Chevilly Larue (FR)

(73) Assignee: L'OREAL, Paris (FR)

(21) Appl. No.: 18/880,931

(22) PCT Filed: Jul. 4, 2023
(86) PCT No.: PCT/EP2023/068410

§ 371 Date: Jan. 3, 2025

(30) Foreign Application Priority Data
Jul. 5, 2022 (FR) 2206824
Jul. 5, 2022 (FR) 2206825

(51) Int. Cl. A61K 8/73; A61K 8/06; A61K 8/37; A61K 8/92; A61Q 1/02

(52) U.S. Cl. CPC A61K 8/731; A61K 8/062; A61K 8/375; A61K 8/922; A61Q 1/02; A61K 2800/43

(57) ABSTRACT

The present invention relates to a liquid cosmetic composition in the form of an emulsion comprising: —from 4% to 25% by weight of ethylcellulose; —at least one natural resin; —at least one first non-volatile oil that is liquid at 25° C., chosen from saturated or unsaturated, linear or branched C₁₀-C₂₆ fatty alcohols; —at least one hydrocarbon-based compound with an ester function, which is solid at ambient temperature; at least one second non-volatile polar hydrocarbon oil, liquid at ambient temperature, different from the first oil and chosen from ester, ether or carbonate oils; or mixtures thereof. The present invention also relates to a process for making up and/or caring for human keratin materials, in particular the skin, the lips, and preferably the lips, which consists in applying the composition according to the invention.

The subject of the present invention is a liquid cosmetic composition provided in the form of an emulsion comprising ethylcellulose, a natural resin, at least one alcohol oil and at least one solid hydrocarbon-based ester compound and/or a particular polar hydrocarbon-based oil, and also a process for making up and/or caring for human keratin materials using same.

At the present time on the market of caring for and/or making up the skin and/or the lips, many products claim good wear property throughout the day, and resistance to external factors such as water, sebum, food, mechanical friction, etc. These “long-lasting” products for the lips, or for the face, that may be used at home, are mainly based on synthetic coating polymers in the presence of organic solvents, mostly volatile oils. For example, compositions are known which comprise a silicone resin as coating agent, for instance trimethyl siloxysilicate (INCI name) or polypropylsilsesquioxane (INCI name) resins, or which comprise silicone polymers such as silicone acrylate dendrimer copolymers (acrylates/polytrimethyl siloxymethacrylate copolymer—INCI name).

In recent years, consumers have become increasingly demanding regarding the composition of their cosmetic products and are in particular seeking to use products with an ever-increasing content of natural ingredients or ingredients of natural origin, ingredients of which the environmental impact is minimized and/or ingredients that are compatible with a wide range of packaging.

The difficulty remains, however, in reconciling these latest trends with the fact that consumers do not, however, want to give up the very high performance to which they have become accustomed regarding the products they already use.

The aim of the present invention is to propose compositions which afford excellent persistence of the expected cosmetic effects, notably the colour of makeup on the skin and the lips, which are resistant to mechanical friction, to meals, to water, to sweat and perspiration, to sebum, to oil, or even to cleaning products, notably makeup-removing products, such as certain micellar waters, or makeup-removing wipes, for example.

In addition, the aim of the present invention is to propose compositions which afford persistence of the expected cosmetic effects, notably the colour of the makeup, combined with an acceptable level of comfort.

These objectives and others are achieved by the present invention, one subject of which is a liquid cosmetic composition in the form of an emulsion, comprising:
4 to 25% by weight, relative to the total weight of the composition, expressed as active ingredient, of ethylcellulose;
at least one natural resin;
at least one first non-volatile oil that is liquid at 25° C., chosen from saturated or unsaturated, linear or branched C₁₀-C₂₆ fatty alcohols;
at least one hydrocarbon-based compound with an ester function, which is solid at ambient temperature; at least one second non-volatile polar hydrocarbon-based oil, liquid at 25° C., different from the first oil and chosen from ester, ether or carbonate oils; or mixtures thereof.

Another subject of the present invention is a process for making up and/or caring for human keratin materials, in particular the skin, the lips, and preferably the lips, which consists in applying the composition according to the invention.

Other aspects and advantages of the present invention will emerge more clearly on reading the description and the examples that follow.

In the context of the present invention, it is indicated that the skin refers to the skin of the face (cheeks, eyelids, eye contour), of the body and of the hands.

The compositions for making up and/or caring for the skin and/or the lips according to the invention are cosmetic compositions. This implies that they advantageously comprise a physiologically acceptable medium. The term “physiologically acceptable” means compatible with the skin and/or the lips, which has a pleasant colour, odour and feel, and which does not cause any unacceptable discomfort (stinging or tautness) liable to discourage the consumer from using this composition.

The term “ambient temperature” means 25° C.

The term “atmospheric pressure” means 760 mmHg, i.e. 1.013×10⁵ pascals.

The composition according to the invention is in the form of an emulsion that is liquid at ambient temperature (25° C.).

The term “liquid” denotes a composition that flows under its own weight after one hour at ambient temperature. Advantageously, the viscosity can be measured according to the following protocol:

The viscosity measurement is performed at 25° C., using a Rheomat RM 180 viscometer equipped with an appropriate spindle (No. 1 to No. 4), the measurement being performed after 10 minutes of rotation of the spindle in the composition, at a shear rate of 200 revolutions/min (rpm).

Preferably, the viscosity at 25° C. of a composition according to the invention is between 0.005 and 18 Pa·s, preferably between 0.01 and 15 Pa·s.

The composition according to the invention can be in the form of an oil-in-water direct emulsion (the continuous phase of the emulsion is the aqueous phase) or in the form of a water-in-oil inverse emulsion (the continuous phase is the lipophilic phrase). Preferably, the composition is in the form of a direct emulsion.

The compositions for making up and/or caring for the skin and/or the lips according to the invention are cosmetic compositions. This implies that they advantageously comprise a physiologically acceptable medium. The term “physiologically acceptable” means compatible with the skin and/or the lips, which has a pleasant colour, odour and feel, and which does not cause any unacceptable discomfort (stinging or tautness) liable to discourage the consumer from using this composition.

### Ethylcellulose

As indicated above, the composition according to the invention comprises at least ethylcellulose.

Ethylcellulose is a cellulose ethyl ether comprising a chain formed from β-anhydroglucose units linked together via acetal bonds. Each anhydroglucose unit contains three replaceable hydroxyl groups, all or some of these hydroxyl groups being able to react according to the following reaction:

RONa+C₂H₅Cl ROC₂H₅+NaCl, in which R represents a cellulose radical.

Total substitution of the three hydroxyl groups would lead, for each anhydroglucose unit, to a degree of substitution of 3, in other words to a content of alkoxy groups, in particular ethoxy groups, of 54.88%.

The ethylcellulose polymers used in a cosmetic composition according to the invention are preferentially polymers with a degree of substitution with ethoxy groups ranging from 2.5 to 2.6 per anhydroglucose unit, in other words comprising a content of ethoxy groups ranging from 44% to 50%.

The average molar mass of the ethylcellulose is preferably chosen such that the viscosity of a 5% by weight solution in a mixture of 80/20 (toluene/ethanol) at 25° C. ranges from 4 to 300 mPa·s, preferably from 5 to 200 mPa·s, for example from 5 to 150 mPa·s. (standard ASTM D 914).

The ethylcellulose used in the composition according to the invention can be in powdered form.

It is, for example, sold under the Ethocel Standard trade names by Dow Chemicals, with in particular Ethocel Standard 7 FP Premium and Ethocel Standard 100 FP Premium. Other commercially available products, such as those sold by Ashland, Inc. under the names Aqualon Ethylcellulose type-K, type-N and type-T, preferably typeN, such as N7, N100, are particularly suitable for performing the invention.

The ethylcellulose used in the composition according to the invention can also be in the form of particles dispersed in an aqueous phase, like a dispersion of latex or pseudolatex type. The techniques for preparing these latex dispersions are well known to those skilled in the art.

The product sold by FMC Biopolymer under the name Aquacoat ECD-30, which is constituted of a dispersion of ethylcellulose in a proportion of about 26.2% by weight in water and stabilized with sodium lauryl sulfate and cetyl alcohol, is most particularly suitable for use as an aqueous dispersion of ethylcellulose.

According to one particular embodiment, the aqueous dispersion of ethylcellulose, in particular the product Aquacoat ECD, may be used in a proportion of from 10% to 90% by weight, in particular from 15% to 60% by weight and preferably from 20% to 50% by weight of ethylcellulose dispersion, relative to the total weight of the composition.

In accordance with a particular embodiment of the invention, the composition comprises ethylcellulose in the aqueous phase of the emulsion (in particular in the form of particles dispersed in an aqueous phase) and comprises ethylcellulose in the lipophilic phase of the composition.

The content of ethylcellulose, expressed as active material, represents more particularly from 4% to 25% by weight, preferably from 5% to 20% by weight, even more preferentially from 6% to 15% by weight, relative to the total weight of the composition.

### Natural Resins

The composition according to the invention comprises at least one natural resin.

Standard IS04618: 2014(fr) defines a resin as a “generally amorphous macromolecular product with a consistency ranging from the solid state to the liquid state”. Natural resins are also considered to be natural adhesives which have the inherent ability to polymerize consistently and predictably by themselves without synthetic chemistry.

Natural resins are virtually exclusively of plant origin (fossil or harvested) and are secreted then exuded by plants for roles of defence, protection and communication within their ecosystem. An exception to this is shellac, which is of animal origin and is secreted by the insect Coccus lacca.

The resin used in the present invention is advantageously chosen from resins of plant origin.

For the purposes of the invention, “natural resin” and in particular “plant resin” means any substance comprising a minimal content of terpenic compounds, i.e. at least 30% by weight of terpenic compounds relative to the total weight of the substance (or material) in question, as defined chemically below, said substance being derived directly or indirectly from the secretion and exudation, mainly by plants (and more rarely by animals), of a substance for roles of defence, protection and communication with their ecosystem.

Advantageously, the natural resin according to the invention is insoluble in water at ambient temperature.

Preferably, the natural resin used in the composition according to the invention has a number-average molecular weight of less than or equal to 10 000 g/mol. The resin preferably has a number-average molecular weight of less than or equal to 10 000 g/mol, particularly ranging from 250 to 10 000 g/mol, preferably less than or equal to 5000 g/mol, particularly ranging from 250 to 5000 g/mol, better still less than or equal to 2000 g/mol, particularly ranging from 250 to 2000 g/mol and even better still less than or equal to 1000 g/mol, particularly ranging from 250 to 1000 g/mol. The number-average molecular weights (Mn) are determined by gel permeation liquid chromatography (THF solvent, calibration curve established with linear polystyrene standards, refractometric detector).

### Thermal Properties

Advantageously, the resins according to the invention are characterized in that they have a softening point, which denotes the temperature of transition from a pseudo-solid state to a plastic state during heating.

Preferably, the resins of the invention have a softening point (or temperature) preferably within the range from 20° C. to 150° C., more preferentially from 30° C. to 100° C., even more preferentially from 40° C. to 90° C.

The softening point is the temperature at which a product reaches a certain degree of softening under standardized conditions. It denotes the temperature of transition from a pseudo-solid state to a plastic state during heating. It can be measured by the ring and ball method (or RBT, ring and ball temperature) for resins according to standard ASTM E284.

Depending on the class thereof, some of the resins according to the invention can also have a melting temperature, preferably of less than 360° C., preferentially less than 190° C., and even more preferentially less than 90° C.

According to a preferred form of the invention, the resins do not have a melting temperature.

The melting point (or melting temperature) of a substance at a given pressure corresponds to the temperature at which the liquid and solid states of this substance can coexist in equilibrium;

Preferably, the resins of the invention have a glass transition temperature preferably within the range from 0° C. to 200° C., more preferentially from 10° C. to 100° C., even more preferentially from 20° C. to 90° C. and even more preferably still from 30° C. to 70° C.

The glass transition (Tg) of a material represents the temperature range through which the material passes from a rubbery state to a vitreous, solid (rigid) state.

The thermal properties, in particular the Mp and Tg of the resins, can be measured by DSC (Differential Scanning Calorimetry), for example by means of a DSC 8000 apparatus from Perkin Elmer, according to:
Protocol 1: Determining melting temperature Mp and crystallization temperature Tc: Starting materials alone or solubilized/dispersed in solvents, stainless steel dishes, sweeping from 5° C. to 90° C., sweep rate of 5° C.·min−1.
Protocol 2: Determining glass transition temperature Tg: measurement on second heating. Aluminium dishes (40 μl) are used, containing the starting materials, a temperature sweep between −100° C. and 150° C. (with isotherms) is carried out in order to observe the glass transition temperature. The temperature ramp applied is 10° C./min for the glass transition temperatures (2 cycles).

### Botanical Definition of Resins

Natural resins of plant origin or animal origin are defined conventionally by Ullmann's Encyclopedia of Industrial Chemistry, “Resins, Synthetic” 2012 WileyVCH Verlag GmbH & Co. KGaA, Weinheim, DOI: 10.1002/14356007.a23_089.pub2. Natural resins particularly include rosins (tall oil rosins, wood or gum originating from tree and plant exudates; wood extracts, or by-products from paper manufacturing), fossil resins such as amber, extracted resins such as asphaltite, shellacs such as those produced by insect secretion, and the main derivatives thereof.

The resins of the invention are preferably of plant origin, particularly from plants or trees.

Fossil resins are (hard and semi-hard) resins collected from the ground, where ancient forests, long since disappeared, once stood. Some fossil resins have undergone considerable changes in their chemical structure due to ageing or maturation, which may have taken thousands of years. The transition from fossil resins to recent resins can vary. They may for example include resins which are both found in fossilized form and are harvested from living plants. Semi-fossil varieties are collected at the base of the trees which produced them (Ullmann's Encyclopedia of Industrial Chemistry, “Resins, Natural” 2012 Wiley-VCH Verlag GmbH & Co. KGaA, Weinheim, DOI: 10.1002/14356007.a23_073) (Techniques de l'Ingénieur [Engineering Techniques], “Résines Naturelles” [Natural Resins], 1982 Bernard Delmond).

Harvested resins are recent (tender). They are harvested from plants which are all living. Depending on their composition, they are subdivided into:
oleoresin: natural solution of resin in an essential oil;
balsam: resin characterized by a high proportion of benzoic and cinnamic acids and esters thereof;
gum: resin composed essentially of polysaccharides;
gum-resin: mixture of resins and hydrophilic gums;
latex: milky composition of organic substances dispersed in an aqueous medium: (Techniques de l'Ingénieur [Engineering Techniques], “Résines Naturelles” [Natural Resins], 1982 Bernard Delmond).

Among the resins, in particular the recent resins, of the invention, resins which are soluble in oils and/or alcohols are preferred to water-soluble forms, such as latices or gums.

According to a preferred mode of the invention, the resins of the invention are harvested resins; these are particularly beneficial according to the invention from an ecological perspective, since they are self-renewing. Preferably, the resins of the invention are recent.

### Chemical Definition of Resins

Chemically, natural resins are complex mixtures of several classes of compounds, the presence and content of which define the glass of the resin (oleoresin, balsam, gum, etc.): essential oils, neutral and acidic constituents and polysaccharides (present exclusively in gums).

The components which characterize resins are the terpenic compounds that they contain, preferably in a content of at least 30% by weight relative to the weight of resin.

“Terpenic compounds” means terpenes, hydrocarbons formed from isoprene having the general formula (C5H8)n, and the numerous derivatives thereof (alcohols, aldehydes, ketones, acids, etc.) comprising a terpene structure (Académie de Montpellier. Les rdsines [Resins]:
https://tice.ac-montpellier.fr/ABCDORGA/Famille/Terpenes.html).

Among the terpenic hydrocarbons, a distinction is made between: monoterpenes of empirical formula C10H16 (n=2), sesquiterpenes of empirical formula C15H24 (n=3), diterpenes (C20H32) (n=4), sesterterpenes (C25H40) (n=5), triterpenes (C30H48) (n=6), tetraterpenes (C40H64) (n=8) and other polyterpenes. Some have an acyclic structure; they comprise a number of double bonds which corresponds to their empirical formula: 3 for C10H16; 5 for C20H32; 7 for C30H48. Others have one or more rings, so a reduced number of double bonds, for example for C10H16 one ring and 2 double bonds, or 2 rings and one double bond.

Advantageously, the resins of the invention contain at least 30% of terpenic compounds, preferably at least 40% by weight of terpenic compounds, preferably at least 50% of terpenic compounds, and even more preferably at least 60% of terpenic compounds by weight relative to the total weight of resin or of resinous substance used as starting material in the composition according to the invention.

Monoterpenic and sesquiterpenic compounds are predominantly volatile compounds, constituting for example essential oils. Polyterpenic compounds derived from terpenes where n is greater than or equal to 4 (such as derivatives of diterpenes and triterpenes) are resinous compounds of a rather solid nature.

According to one preferred embodiment of the invention, the resins comprise at least 10%, preferably at least 20% by weight, preferably at least 30% by weight, preferably at least 35% by weight, of polyterpenic compounds, i.e. of compounds derived from terpenes where n is greater than or equal to 4, relative to the total weight of resin, representing 100%. Thus, preference is given to resins having a fraction which is solid at ambient temperature (25° C.). Advantageously, said resins used according to the invention are not volatile.

Advantageously, the polyterpenic compounds of the resins or resinous substances used in the composition of the invention are predominantly (to more than 50% by weight relative to the total weight of polyterpenes) derived from diterpenes and/or from triterpenes.

According to one preferred embodiment of the invention, the resins comprise less than 70% by weight of monoterpenic or sesquiterpenic compounds, i.e of compounds derived from terpenes where n is less than 4, relative to the total weight of resin, representing 100%; preferably, said resins comprise less than 60% by weight, preferably less than 50% by weight, preferably less than 30% by weight, preferably less than 15% by weight, of monoterpenic or sesquiterpenic compounds derived from terpenes where n is less than 4 relative to the total weight of resin, representing 100% Thus, for the compositions of the invention, preference is given to limiting the use of the most volatile resins, since they are less effective in terms of the wear property of a cosmetic film.

A non-exhaustive list of terpenic compounds that may be contained in the natural resins of the invention has been produced. It lists families of terpenic compounds, subdivided on the bases of the characteristic groups (alcohol function, ketone function, acid function, etc.) of each compound (listed below).

### Examples of Monoterpenic Compounds

Advantageously, the monoterpene compounds of the resin are chosen from α-pinene, β-pinene, 3-carene, camphene, dipentene, P-cymene, B-myrcene, α-phellandrene, sabinene, α-thujene, limonene, octyl ethanoate, neryl ethanoate, bornyl ethanoate, geranyl ethanoate, α-terpineol, cineol, linalool, borneol, derivatives thereof and mixtures thereof.

### Examples of Sesquiterpenic Compounds

Advantageously, the sesquiterpene compounds of the resin are chosen from: alphacopaene, β-caryophyllene, β-bisabolene, β-gurjunene, alpha-gurjunene, alloaromadendrene, β-bourbonene, delta-cadinene, alpha-guaiene, α-elemene, β-elemene, d-elemene, α-copaene, α-selinene, β-selinene, β-bourbonene, lindestrene, furanoeudesma-1,3-diene, α-cubebene, farnesol, α-elemol, viridiflorol, t-cadinol, β-elemol, germacrone, curzerenone, derivatives thereof and mixtures thereof.

### Examples of Diterpenic Compounds

Advantageously, the diterpenic compounds of the resin are chosen from: Abietic acid, pimaric acid, sandaropimaric acid, comunic acid, levopimaric acid, pallustric acid, isopimaric acid, dehydroabietic acid, neoabietic acid, agathic acid, cembrene A, cembrene C, isocembrene, Vercilla-4(20),7,11-triene, incensole, totarol, sandaracopimarinol, cembrenol, derivatives thereof and mixtures thereof.

### Examples of Triterpenic Compounds

Advantageously, the triterpenic compounds of the resin are chosen from: 3β,20(S)-dihydroxydammar-24-ene, dammarenolic acid, dammardienone, hydroxydammarenone (I or II), dammarenediol I (or II), dammadienol, 11-keto-β-boswellic acid (KBA), 11-keto-β-boswellic acid acetate (AKBA), β-boswellic acid, ursolic acid, mangiferonic acid, benthamic acid, ursolic aldehyde, α-amyrenone, α-amyrin, Bamyrin, uvaol, oleanolic acid, oleanonic acid, moronic acid, oleanonic aldehyde, acetyl-lupeolic acid, lupeolic acid, lupeol,
betulonal, hydroxyhopanone, derivatives thereof and mixtures thereof.

According to a first embodiment of the invention, the resin(s) used according to the present invention contain at least one diterpenic compound, preferably derived from abietic acid, which is natural or chemically modified.

According to a second embodiment of the invention, as an alternative or indeed in addition to the first, the resin(s) used according to the present invention contain at least one triterpenic compound, preferably chosen from the following triterpenic compounds: alpha-amyrin, beta-amyrin, alpha-amyrone, beta-amyrone, dammadienone, dammadienol, ursolic aldehyde, hydroxyhopanone, oleanonic aldehyde, ursolic acid, oleanonic acid, oleanolic acid, and mixtures thereof. The total content of triterpenic compounds, particularly the content of those preferred above, in the resin used according to the invention is advantageously at least 10%, preferably at least 20%, even more preferentially at least 30%, and preferably at least 35% by weight relative to the total weight of the natural resin. Mention may particularly be made of the frankincense resins, such as the Protium heptaphyllum resins or else Shorea robusta resins, containing such triterpenic compounds.

The chemical composition of a resin may be analysed by conventional techniques known to those skilled in the art, such as gas chromatography GC analysis, chromatographic analysis with flame ionization detection, referred to as GC-FID, or GC/MS analysis, which consists in using a mass spectrometer coupled to a gas chromatograph; preferably by GC-FID. The following paper mentions these common methods: “Développements méthodologiques en TLC/MALDITOF M S et GC/MS pour l'analyse des composés terpénoïdes présents dans les résines végétales” [Methodological developments in TLC/MALDITOF MS and GC/MS for the analysis of terpenoid compounds present in plant resins], https://tel.archives-ouvertes.fr/tel-01581308.

Definition of the Resins by their Origin:

Advantageously, the natural resin(s) according to the invention are chosen from: a) acaroid resins, b) ambers, c) asphaltite and gilsonite, d) Peru balsam, e) Tolu balsam, f) benzoin resins, g) Canada balsam, h) copal resins (particularly kauri copal resins, copal resins from Manilla, West African copals such as Congolese, Angolan or Camaroonian copals, East African copals such as Zanzibari or Madagascan copals, South American copals such as Brazilian or Colombian copals), i) damars, j) elemis, k) frankincenses, l) galbanums, m) labdanums, n) mastics, o) myrrh, p) sandarac, q) shellacs, r) styrax (storax), s) Venice turpentine (larch, turpentine essence), t) rosins, particularly rosin, rosinate and tall oils, v) resins extracted from plant waxes, and mixtures of these resins.

Preferably, the natural resin(s) used according to the invention are chosen from the frankincense resins (k) and the resins extracted from plant waxes (v). It is understood that the resin(s) of the invention can be esterified, salified, adducts, phenol-modified, and/or dimerized and additionally hydrogenated.

### Frankincense Resins (Oliban)

Frankincenses are present in the United Arab Emirates, Oman, Somalia, Ethiopia and Eastern India. Frankincense resins are recent and are taken from the Boswellia carterii tree frankincense. Amazonian frankincense resins also exist. The bark is intentionally injured in order to obtain a milky extract which is recovered after drying. Preferably, the resin(s) of the invention are chosen from frankincenses, particularly Amazonian frankincenses.

Frankincense resins are pale yellow and form irregular round or globular beads. They generally contain from 20% to 40% by weight (approx. 33%) of boswellic acid (C₃₂H₅₂O₄). Frankincenses have an acid number of between 30% and 50% (indirect) and are moderately soluble in ethanol in basic medium.

According to one particular embodiment of the invention, the resin(s) of the invention are chosen from frankincenses, particularly Amazonian frankincense resins sold under the name Protium Heptaphyllum resin, or Protium Resin, or White Breu Resin, and frankincense resins originating from the sal tree, Shorea robusta.

Advantageously, the resin(s) are in a mixture with one or more fatty substances as defined below according to the invention, preferably chosen from volatile or nonvolatile oils. Mention may be made for example of Shorea robusta resin with sunflower seed oil (Shorea Robusta Resin, Helianthus Annuus (Sunflower) Seed Oil, tocopherol: 50-75% by weight of shorea robusta resin, 25-50% by weight of sunflower seed oil) sold under the name Kahlresin 6720, and Shorea robusta resin with octyldodecanol (Shorea Robusta Resin and Octyldodecanol; 50-70% by weight Shorea Robusta Resin, 30-50% by weight of octyldodecanol) sold by Kahlresin 6720.

Resins Extracted from Plant Waxes

Natural plant waxes per se are not considered resins. Although they are among the substances secreted/excreted by plants and naturally contain a very low content of resins, they contain less than 30% by weight of terpenes relative to the total weight of wax. For example, carnauba wax is secreted naturally by the leaves of a palm tree, Copernica cerifera, to prevent the leaves from dehydrating. Candelilla wax is obtained from a shrub named Euphorbia antisyphilitica which originates from northern Mexico. The wax protects the plant from its environment and prevents excessive evaporation. For example, candelilla wax is composed mainly of hydrocarbons (approximately 50%, chains from 29 to 33 carbon atoms), of higher-molecular-weight esters (20% to 29%), of free acids (7% to 9%) and of resins (12-14%, mainly triterpenic esters).

Nevertheless, the definition of “natural resins” for the purposes of the present invention also includes resins resulting from plant waxes, when they have been concentrated, isolated or extracted beforehand from these waxes, as long as the resinous or terpenic ingredient in question contains the minimal content of terpenes (30% by weight relative to the total weight of the ingredient) required by the present invention. Mention may particularly be made of candelilla resin (pure 100% resin, extracted from the corresponding wax) having the INCI name: Euphorbia Cerifera (Candellila) Wax Extract, sold under the name Candelilla Resin E-1 by Japan Natural Products. Document WO2013/147113 A1 also refers to carnauba resin, a terpenic resin extracted from carnauba wax which has similar physical properties to those of the natural resins conventionally described, such as a softening temperature and not a melting temperature, which distinguishes a resin from a wax.

According to one preferred embodiment of the invention, the resin(s) are chosen from Euphorbia Cerifera (Candellila) Wax Extracts, Protium Heptaphyllum Resins, Shorea Robusta Resins, and also a mixture thereof, and preferably from Euphorbia Cerifera (Candellila) Wax Extracts, Shorea Robusta Resins, and also a mixture thereof.

Advantageously, the resin(s) is (are) present in the composition of the invention in a content within the range from 0.5% to 15%, preferably from 0.5% to 10%, even more preferentially from 0.5% to 8%, by weight relative to the total weight of the composition.

### First Oil

As indicated above, the composition according to the invention comprises, as first non-volatile oil that is liquid at 25° C., saturated or unsaturated and linear or branched C₁₀-C₂₆ fatty alcohols, preferably monoalcohols.

Advantageously, the C₁₀-C₂₆ alcohols are fatty alcohols, which are preferably branched when they comprise at least 16 carbon atoms. Preferably, the fatty alcohol comprises from 10 to 24 carbon atoms and more preferentially from 12 to 22 carbon atoms. As particular examples of fatty alcohols that may preferably be used, mention may be made especially of lauryl alcohol, isostearyl alcohol, oleyl alcohol, 2-butyloctanol, 2-undecylpentadecanol, 2-hexyldecyl alcohol, isocetyl alcohol and octyldodecanol, and mixtures thereof. According to one advantageous embodiment of the invention, the alcohol is chosen from octyldodecanol.

More particularly, according to a first embodiment of the present invention, the content of first oil ranges from 0.5% to less than 20% by weight, more particularly from 2% to 15% by weight, relative to the total weight of the composition.

More particularly, according to a second embodiment of the present invention, the content of first oil ranges from 20% to 60% by weight, more particularly from 20% to 40% by weight, relative to the total weight of the composition

### Second Oil

The composition according to the invention can optionally comprise at least one specific second non-volatile hydrocarbon-based oil that is liquid at 25° C. and atmospheric pressure (1.013×10⁵ Pa).

The term “non-volatile oil” means an oil, the vapour pressure of which at 25° C. and atmospheric pressure is non-zero and is less than 2.66 Pa and more particularly less than 0.13 Pa. By way of example, the vapour pressure may be measured according to the static method or via the effusion method by isothermal thermogravimetry, depending on the vapour pressure of the oil (standard OCDE 104).

The composition according to the invention comprises, as second oil, at least one oil chosen from:
ethers of formula ROR′ or carbonates of formula RO(CO)OR′, in which formulae, identical or not, the R and R′ groups represent a saturated or unsaturated, branched or unbranched, hydrocarbon-based group comprising at most 16 carbon atoms, preferably a C₃-C₁₆ group;
hydroxylated or non-hydroxylated plant oils;
optionally hydroxylated ester oils comprising from 1 to 4 ester functions, of which at least one of them, which is linear or branched, saturated, unsaturated or aromatic, comprises at least 10 carbon atoms;
liquid polyesters derived from the reaction of a monounsaturated or polyunsaturated acid dimer; the fatty acid comprising from 16 to 22 carbon atoms;
and also mixtures thereof.

Preferably, the second oil is chosen from:
dicaprylyl ether;
dipropyl carbonate, diethylhexyl carbonate, dicaprylyl carbonate, and C₁₄-C₁₅ dialkyl carbonate;
castor oil, olive oil, jojoba oil, ximenia oil, pracaxi oil, wheat germ oil, maize oil, sunflower oil, shea oil, sweet almond oil, macadamia oil, apricot kernel oil, soybean oil, rapeseed oil, peanut oil, cottonseed oil, alfalfa oil, poppy oil, red kuri squash oil, sesame oil, pumpkin oil, avocado oil, hazelnut oil, grape seed oil, blackcurrant oil, argan oil, evening primrose oil, millet oil, barley oil, linseed oil, quinoa oil, rye oil, safflower oil, candlenut oil, passionflower oil, musk rose oil, the liquid fraction of shea butter and the liquid fraction of cocoa butter, and mixtures thereof;
2-ethylhexyl palmitate, 2-octyldecyl palmitate, octyldodecyl neopentanoate, 2-octyldodecyl stearate, butyl stearate, 2-octyldodecyl erucate, C₁₂ to C₁₅ alkyl benzoates, 2-octyldodecyl benzoate, isocetyl isostearate, isostearyl isostearate, isononyl isononanoate, isopropyl palmitate, hexyl laurate, 2-hexyldecyl laurate, isopropyl myristate, 2-octyldodecyl myristate, diisostearyl malate, neopentylglycol dicaprate, glyceryl tri(2-decyltetradecanoate), capric/caprylic acid triglycerides, C₁₈₋₃₆ acid triglycerides, glyceryl triheptanoate, glyceryl trioctanoate, glyceryl tri(2-decyltetradecanoate), triisostearyl citrate, tridecyl stearate, tridecyl trimellitate, pentaerythrityl tetrapelargonate, pentaerythrityl tetraisostearate, pentaerythrityl tetraisononanoate, pentaerythrityl tetra(2-decyltetradecanoate); isostearyl lactate, octyl hydroxystearate, octyldodecyl hydroxystearate;
polyesters with the following INCI names: dilinoleic acid/butanediol copolymer, dilinoleic acid/propanediol copolymer, dimer dilinoleyl dilinoleate;
and also mixtures thereof.

Preferably, the second oil is chosen from dicaprylyl ether; dicaprylyl carbonate; nonhydroxylated plant oils, such as, for example, olive oil; castor oil; capric/caprylic acid triglycerides, isocetyl isostearate, isononyl isononanoate, neopentylglycol dicaprate, tridecyl stearate, pentaerythrityl esters, such as, in particular, pentaerythrityl tetraisostearate, and also mixtures thereof.

The content of second oil, if the composition comprises any, ranges from 2% to 35% by weight, more particularly from 5% to 30% by weight, relative to the total weight of the composition.

Solid Hydrocarbon-Based Compound with an Ester Function

The composition according to the invention can optionally also comprise at least one hydrocarbon-based compound that is solid at ambient temperature, comprising an ester function. For the purposes of the invention, this means that said compound comprises at least one ester function (—COOR).

The term “hydrocarbon-based compound” conventionally denotes a compound formed essentially from, or even constituted of, carbon and hydrogen atoms, and optionally oxygen and nitrogen atoms, and not containing any other heteroatoms, such as silicon, fluorine, phosphorus, etc. atoms. Advantageously, the solid hydrocarbon-based compounds used according to the invention contain only carbon, hydrogen, oxygen atoms.

The solid compound can be chosen from waxes.

More particularly, the wax is a lipophilic compound that is solid at a temperature greater than 22° C., preferably greater than or equal to 25° C., with a reversible solid/liquid change of state, having a melting point in particular of greater than or equal to 30° C., even more particularly greater than or equal to 45° C. Advantageously, the melting point is less than or equal to 90° C., more particularly less than or equal to 80° C. and preferably less than or equal to 70° C.

The melting point of a solid fatty substance can be measured using a differential scanning calorimeter (DSC), for example the calorimeter sold under the name DSC Q100 by TA Instruments with the TA Universal Analysis software.

The measuring protocol is as follows:

A sample of approximately 5 mg of solid fatty substance is placed in a “hermetic aluminium capsule” crucible.

The sample is subjected to a first temperature rise extending from 20° C. to 120° C., at a heating rate of 2° C./minute up to 80° C., it is then left at the 100° C. isotherm for 20 minutes, is then cooled from 120° C. to 0° C. at a cooling rate of 2° C./minute, and finally is subjected to a second temperature rise extending from 0° C. to 20° C. at a heating rate of 2° C./minute.

The melting point value of the solid fatty substance is the value of the top of the most endothermic peak observed of the melting curve, representing the variation in the difference in power absorbed as a function of the temperature.

The solid compound can be chosen from pasty compounds.

For the purposes of the present invention, the term “pasty compound” means a lipophilic compound with a reversible solid/liquid change of state, and including at a temperature of 20° C. a liquid fraction and a solid fraction. Thus, a pasty compound can exhibit a starting melting point of less than 20° C. Furthermore, the pasty compound can have, in the solid state, an anisotropic crystalline organization. The melting point of the pasty fatty substance is determined according to the same principle as that described in detail above for the waxes.

In the case of a pasty compound, the measuring protocol is, however, as follows:

A sample of 5 mg of pasty fatty substance placed in a crucible is subjected to a first temperature rise passing from −20° C. to 100° C., at a heating rate of 10° C./minute, it is then cooled from 100° C. to −20° C. at a cooling rate of 10° C./minute and it is finally subjected to a second temperature rise passing from −20° C. to 100° C. at a heating rate of 5° C./minute.

The melting point of the pasty fatty substance is the value of the temperature corresponding to the top of the peak on the curve representing the variation in the difference in power absorbed as a function of the temperature.

It should be noted that the liquid fraction by weight of the pasty fatty substance at ambient temperature is equal to the ratio of the heat of fusion consumed at ambient temperature to the heat of fusion of the pasty fatty substance.

The heat of fusion of the pasty fatty substance is the heat consumed by said substance in order to pass from the solid state to the liquid state. The pasty fatty substance is said to be in the solid state when all of its mass is in crystalline solid form. The pasty fatty substance is said to be in the liquid state when all of its mass is in liquid form.

The heat of fusion of the pasty fatty substance is the amount of energy required to make the pasty fatty substance change from the solid state to the liquid state. It is expressed in J/g. The heat of fusion of the pasty fatty substance is equal to the area under the curve of the thermogram obtained.

Among the solid hydrocarbon-based compounds that may be used in the context of the present invention, mention may be made in particular of:
plant butters,
totally or partially hydrogenated plant oils,
plant waxes,
beeswax, synthetic beeswax, lanolin wax,
esters of hydrogenated castor oil and of C₁₆-C₂₂ fatty acids,
saturated or unsaturated, linear or branched, optionally monohydroxylated or polyhydroxylated, C₁₂-C₃₀, preferably C₁₂-C₁₈, optionally hydrogenated fatty acid triglycerides,
the waxes obtained by hydrogenation of plant oil which are esterified with fatty alcohols, such as, in particular, lauryl, stearyl, cetyl or behenyl alcohol,
the esters of formula R₁COOR₂ in which R₁ and R₂ represent linear, branched or cyclic aliphatic chains in which the number of atoms ranges from 10 to 50, which may contain a heteroatom, in particular oxygen,
the polyesters derived from the condensation of a linear or branched C₆-C₁₀ dicarboxylic acid and of an ester of diglycerol and of optionally hydroxylated, linear or branched C₆-C₂₀ monocarboxylic acids,
polyesters obtained from an acid dimer, said acid being unsaturated and comprising 16 to 24 carbon atoms, and from at least one alcohol or polyol,
mixtures thereof.

Preferably, this or these pasty hydrocarbon-based compound(s) are chosen from:
plant butters, for instance mango butter, such as the product sold under the reference Lipex® 203 by Aarhuskarlshamn, shea butter, in particular the product of which the INCI name is Butyrospermum parkii Butter, such as the product sold under the reference Sheasoft® by Aarhuskarlshamn, cupuacu butter (Rain Forest RF3410 from Beraca Sabara), murumuru butter (Rain Forest RF3710 from Beraca Sabara), cocoa butter; babassu butter such as the product sold under the name Cropure® Babassu by Croda, and also orange wax, for example the product sold under the reference Orange Peel Wax by Koster Keunen,
totally or partially hydrogenated plant oils, for instance hydrogenated soybean oil, hydrogenated coconut kernel oil, hydrogenated rapeseed oil, mixtures of hydrogenated plant oils such as the mixture of hydrogenated soybean, coconut kernel, palm and rapeseed plant oil, for example the mixture sold under the reference Akogel® by Aarhuskarlshamn (INCI name Hydrogenated Vegetable Oil), the trans-isomerized partially hydrogenated jojoba oil manufactured or sold by Desert Whale under the commercial reference Iso-Jojoba-50®, partially hydrogenated olive oil, for instance the compound sold under the reference Beurrolive by Soliance,
waxes of plant origin, such as sunflower wax, carnauba wax, candelilla wax, rice bran wax, ouricury wax, esparto grass wax, berry wax, cork fibre wax, sugar cane wax, Japan wax, sumac wax, montan wax, orange and lemon waxes, laurel wax, and mixtures thereof,
beeswax, synthetic beeswax, lanolin wax,
esters of hydrogenated castor oil and of C₁₆-C₂₂ fatty acids, in particular of isostearic acid, such as the compound having the INCI name Hydrogenated Castor Oil Isostearate, for example Salacos HCIS (V-L), sold by Nisshin Oil,
saturated or unsaturated, linear or branched, optionally monohydroxylated or polyhydroxylated, optionally hydrogenated, in particular C₁₂-C₃₀, fatty acid triglycerides. Mention may be made for example of triglycerides of optionally hydrogenated (totally or partially), saturated or unsaturated, linear or branched, optionally monohydroxylated or polyhydroxylated, preferably C₁₂-C₁₈ fatty acids; for instance the glycerides of saturated C₁₂-C₁₈ fatty acids sold under the name Softisan 100® by Cremer Oleo (INCI name: Hydrogenated Coco-Glycerides); triglycerides of a saturated, optionally hydroxylated, C₁₆-C₃₀ carboxylic acid; for example, mention may be made of trihydroxystearin (or glyceryl trihydroxystearate), tristearin (or glyceryl tristearate), tribehenin (or glyceryl tribehenate), alone or as a mixture. Among the suitable compounds, mention may be made of triesters of glycerol and of 12-hydroxystearic acid, or hydrogenated castor oil, for instance Thixcin R and Thixcin E sold by Elementis Specialties,
the waxes obtained by hydrogenation of plant oil which are esterified with fatty alcohols, such as, in particular, lauryl, stearyl, cetyl or behenyl alcohol. For example, mention may be made of the waxes obtained by hydrogenation of castor oil esterified with cetyl alcohol, such as those sold under the names Phytowax Castor 16L64® and 22L73® by Sophim. Such waxes are described in patent application FR-A-2 792 190. Also suitable are waxes obtained by hydrogenation of olive oil esterified with stearyl alcohol, such as for example those sold under the name Phytowax Olive 18 L 57,
the esters of formula R₁COOR₂ in which R₁ and R₂ represent linear, branched or cyclic aliphatic chains in which the number of atoms ranges from 10 to 50, which may contain a heteroatom, in particular oxygen, In particular, use may be made, as ester wax, of a C₂₀-C₄₀ alkyl (hydroxystearyloxy)stearate (the alkyl group comprising from 20 to 40 carbon atoms), alone or as a mixture, or a C₂₀-C₄₀ alkyl stearate. Such waxes are sold in particular under the names Kester Wax K 82 P®, Hydroxypolyester K 82 P®, Kester Wax K 80 P® or Kester Wax K82H by Koster Keunen. Use may also be made of mixtures of esters of C₁₄-C₁₈ carboxylic acids and of alcohols, such as the products Cetyl Ester Wax 814 from Koster Keunen, SP Crodamol MS MBAL or Crodamol MS PA from Croda, or Miraceti from Laserson.

Use may also be made of a glycol and butylene glycol montanate (octacosanoate), such as the wax Licowax KPS Flakes (INCI name: glycol montanate) sold by Clariant,
natural or synthetic polyalkylenated or polyglycerolated hydrocarbon-based waxes, of animal or plant origin; the number of (C₂-C₄) oxyalkylene units can range from 2 to 100, the number of glycerol units can range from 1 to 20. By way of examples, mention may be made of polyoxyethylenated beeswaxes, such as PEG-6 beeswax or PEG-8 beeswax; polyoxyethylenated carnauba waxes, such as PEG-12 carnauba; polyoxyethylenated or polyoxypropylenated and hydrogenated or non-hydrogenated lanolin waxes, such as PEG-30 lanolin or PEG-75 lanolin; PPG-5 lanolin wax glyceride; polyglycerolated beeswaxes, in particular polyglyceryl-3 beeswax, the Acacia Decurrens/Jojoba/Sunflower Seed Wax/Polyglyceryl-3 Esters mixture, polyglycerolated plant waxes, such as mimosa, jojoba or sunflower waxes, and mixtures thereof (Acacia Decurrens/Jojoba/Sunflower Seed Wax Polyglyceryl-3 Esters),
polyesters obtained from the condensation of a linear or branched C₆-C₁₀ dicarboxylic acid and of an ester of diglycerol and of optionally hydroxylated, linear or branched C₆-C₂₀ monocarboxylic acids, notably such as the ester obtained by condensation of adipic acid and a mixture of diglycerol esters with a mixture of C₆-C₂₀ fatty acids such as caprylic acid, capric acid, stearic acid, isostearic acid and 12-hydroxystearic acid, and having the INCI name Bis-Diglyceryl Polyacyladipate-2. This type of compound is notably sold under the reference Softisan® 649 by Cremer Oleo,
polyesters obtained from an acid dimer, said acid being unsaturated and comprising 16 to 24 carbon atoms, and from at least one alcohol or polyol, for instance:
esters of diol dimer (for example of dilinoleyl alcohol) and of dilinoleic acid, the hydroxyl groups of which are esterified with a mixture of phytosterols, of behenyl alcohol and of isostearyl alcohol, for example the ester sold under the name Plandool G by Nippon Fine Chemical (INCI name: Bis-Behenyl/Isostearyl/Phytosteryl Dimer Dilinoleyl Dimer Dilinoleate);
esters of dilinoleic acid and of a mixture of phytosterols, of isostearyl alcohol, of cetyl alcohol, of stearyl alcohol and of behenyl alcohol, for example the ester sold under the name Plandool H or Plandool S by Nippon Fine Chemical (INCI name: Phytosteryl/Isostearyl/Cetyl/Stearyl/Behenyl Dimer Dilinoleate);
esters of hydrogenated castor oil and of dilinoleic acid, such as those sold under the names Risocast-DA-L or Risocast-DA-H by Kokyu Alcohol Kogyo (INCI name: Hydrogenated Castor Oil Dimer Dilinoleate).

Preferably, the solid hydrocarbon-based compound is chosen from saturated or unsaturated, linear or branched, optionally mono hydroxylated or poly-hydroxylated, optionally hydrogenated, preferably C₁₂-C₃₀, preferably C₁₂-C₁₈, fatty acid triglycerides, such as Hydrogenated Coco-Glycerides.

Advantageously, according to a first embodiment, if present, the content of hydrocarbon-based compound(s) that are solid at ambient temperature is at least 15% by weight, more particularly at least 20% by weight, and preferably between 20% and 60% by weight relative to the total weight of the composition.

Advantageously, according to a second variant, if present, the content of solid hydrocarbon-based compound(s) at room temperature is less than 15% by weight, between 0 (limit included) and less than 15% by weight, preferably between 0 and 10% by weight (limits included) relative to the total weight of the composition.

### Additional Oils

### Volatile Hydrocarbon-Based or Silicone Oils

The composition according to the invention may optionally comprise at least one volatile hydrocarbon-based or silicone oil, or a mixture thereof.

The term “volatile oil” is understood to mean an oil having a vapour pressure of greater than or equal to 2.66 Pa, at ambient temperature (25° C.) and atmospheric pressure, ranging in particular from 2.66 Pa to 40 000 Pa, in particular ranging to 13 000 Pa and preferably to 1300 Pa.

The volatile hydrocarbon-based oils are preferably chosen from non-polar hydrocarbon-based oils.

The term “non-polar” is understood to mean a hydrocarbon-based oil constituted of carbon atoms and hydrogen atoms (hydrocarbon-type compound).

The term “silicone oil” is understood to mean an oil comprising at least one silicon atom.

With regard to the volatile hydrocarbon-based oils, they are chosen more particularly from volatile hydrocarbon-based oils containing from 8 to 16 carbon atoms, and mixtures thereof, and in particular:
branched C₈-C₁₆ alkanes, such as C₈-C₁₆ isoalkanes (also known as isoparaffins), isododecane, isodecane or isohexadecane, and for example the oils sold under the Isopar or Permethyl trade names,
linear alkanes, for example C₁₁-C₁₆ alkanes, alone or as mixtures, and
mixtures thereof.

Mention may in particular be made, as volatile silicone oils, of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and mixtures thereof.

If the composition comprises any, the content of volatile hydrocarbon-based or silicone oil(s) is preferably less than or equal to 10% by weight, preferably less than or equal to 5% by weight, relative to the total weight of the composition, and advantageously the composition does not comprise any volatile oil.

### Non-Polar Non-Volatile Hydrocarbon-Based Oils

According to another particular embodiment, the composition according to the invention may comprise at least one non-polar non-volatile hydrocarbon-based oil.

By way of examples, the non-polar non-volatile hydrocarbon-based oil can be chosen from liquid paraffin, squalane, isoeicosane, non-volatile mixtures of saturated linear hydrocarbons, hydrogenated or non-hydrogenated polybutenes, hydrogenated or nonhydrogenated polyisobutenes, hydrogenated or non-hydrogenated polydecenes, decene/butene copolymers, polybutene/polyisobutene copolymers, and mixtures thereof.

Preferably, the composition according to the invention comprises from 0% to 10% by weight, preferably from 0.1% to 5% by weight, relative to the total weight of the composition, of non-polar non-volatile hydrocarbon-based oil(s). Preferably, the composition according to the invention does not comprise any non-polar non-volatile hydrocarbon-based oil(s).

### Silicone Oils

The composition according to the invention may optionally comprise at least one non-volatile silicone oil.

Mention may in particular be made, among non-volatile silicone oils, of dimethicones, dimethiconols, trimethyl pentaphenyl trisiloxanes, tetramethyl tetraphenyl trisiloxanes, diphenyl dimethicones, trimethylsiloxyphenyl dimethicones, phenyl trimethicones, diphenylsiloxy phenyl trimethicones; and also mixtures thereof (INCI names).

In accordance with one embodiment of the present invention, if such oils were present, the content thereof would be less than or equal to 10% by weight, more preferentially less than or equal to 4% by weight, more particularly less than or equal to 2% by weight, and advantageously less than or equal to 1% by weight, relative to the total weight of the composition. Advantageously, the composition according to the invention is free of silicone oils.

### Aqueous Phase

The composition according to the invention comprises water.

More particularly, the composition according to the invention comprises at least 15% by weight, in particular from 20% to 60% by weight and especially from 25% to 50% by weight of water, relative to the total weight of the composition.

The composition may also comprise at least one monoalcohol containing from 1 to 5 carbon atoms, which is preferably saturated, preferably such as ethanol or isopropanol.

If the composition comprises them, the content of monoalcohol ranges between 0% and 10% by weight relative to the total weight of the composition.

The composition can also optionally comprise at least one polyol that is liquid at 25° C. and atmospheric pressure (1.013×10⁵ Pa).

The term “polyol” denotes any organic molecule comprising at least two hydroxyl groups (or free hydroxyl groups).

More particularly, the liquid polyol(s) are chosen from saturated or unsaturated, linear or branched, C₂-C₈, more particularly C₂-C₆, hydrocarbon-based compounds comprising at least two hydroxyl functions, preferably comprising from 2 to 6 hydroxyl groups. Advantageously, the liquid polyol can, for example, be chosen from ethylene glycol, pentaerythritol, trimethylolpropane, propylene glycol, dipropylene glycol, 1,3-propanediol, butylene glycol, 1,3-butylene glycol, isopentyldiol, pentylene glycol, hexylene glycol, glycerol, ethylhexyl glycerol, diglycerol and mixtures thereof. Preferably, the polyol is chosen from glycerol, propylene glycol, 1,3-butylene glycol, dipropylene glycol, diglycerol and mixtures thereof; even more advantageously, glycerol.

Preferably, if the composition comprises at least one liquid polyol as defined above, its content ranges from 0% to 20% by weight, relative to the total weight of the composition.

The composition according to the invention preferentially comprises at least 20% by weight of aqueous phase (water+C₁-C₅ monoalcohol(s)+liquid polyol(s)), in particular from 20% to 60% by weight and especially from 25% to 50% by weight, relative to the total weight of the composition.

### Surfactants

The composition according to the invention can optionally comprise at least one nonionic or anionic, preferably hydrocarbon-based, surfactant.

Those skilled in the art, by means of their general knowledge, are able to determine whether it is necessary to use one or more surfactants.

As a guide, if the composition comprises any surfactant(s), the content thereof represents from 0.01% to 6% by weight relative to the total weight of the composition.

### Non-Ionic Surfactants

The choice of surfactant(s) depends on the nature of the desired emulsion.

In particular, if an oil-in-water emulsion is envisaged, the surfactant(s) will preferably be at least chosen from non-ionic hydrocarbon-based or silicone surfactants with an HLB (hydrophilic-lipophilic balance) value within the meaning of Griffin as defined in J. Soc. Cosm. Chem. 1954 (volume 5), pages 249-256, of greater than or equal to 8, and preferably at least from among hydrocarbon-based surfactants.

If a water-in-oil emulsion is moreover envisaged, then the surfactant(s) will preferably be chosen from non-ionic hydrocarbon-based or silicone surfactants with an HLB value (as defined above) of less than than 8, or mixtures thereof.

The non-ionic surfactant(s) can be chosen in particular from (poly)oxyalkylenated alcohols, (poly)oxyalkylenated ethers, (poly)oxyalkylenated esters, (poly)oxyalkylenated or non-(poly)oxyalkylenated sorbitan esters, (poly)oxyalkylenated or non-(poly)oxyalkylenated sorbitan ethers, alkyl(poly)glucosides, sugar esters, (poly)oxyalkylenated or non-(poly)oxyalkylenated glycerol esters, (poly)oxyalkylenated or non-(poly)oxyalkylenated glyceryol ethers, and also mixtures thereof. The term “(poly)oxyalkylenated” denotes an oxyethylene and/or oxypropylene unit, the number of units ranging between 1 and 200.

Among the (poly)oxyethylenated alcohols, mention may be made of:
polyoxyethylenated fatty alcohols of R(O—CH₂—CH₂)o-OH type, R representing a C₈-C₃₀ alkyl radical, o representing an average integer ranging from 2 to 50, in particular chosen from ceteth-2, ceteth-5, ceteth-7, ceteth-10, ceteth-15, ceteth-20, ceteth-23, ceteth-25, ceteth-40, isoceteth-20, laureth-2, laureth-3, laureth-4, laureth-7, laureth-12, laureth-23, oleth-2, oleth-5, oleth-7, oleth-10, oleth-12, oleth-20, oleth-25, oleth-50, deceth-3, deceth-5, beheneth-10, beheneth 100, steareth-2, steareth-6, steareth-10, steareth-20, steareth-21, steareth-40, steareth-100, ceteareth-7, ceteareth-10, ceteareth12, ceteareth-15, ceteareth-20, ceteareth-25, ceteareth-30, ceteareth-33, pareth-3, pareth-23, C₁₂-C₁₅ pareth-3, C₁₂-C₁₃ pareth-4, C₁₂-C₁₃ pareth-23, coceth-7, trideceth-3, trideceth-4, trideceth-5, trideceth-6, trideceth-7, trideceth-10, trideceth-12, phytosterol 30 EO, and mixtures thereof;
polyoxyethylenated and oxypropylenated alcohols of the type: R—(O—C(CH₃)H—CH₂)o-(O—CH₂—CH₂)p-OH; R representing a C₄-C₃₀ alkyl radical, o and p, independently of each other, representing an average integer ranging from 1 to 50. Preferably, the compound Y is chosen from the compounds of which the INCI name is as follows: PPG-26-buteth-26, PPG-12-buteth-16, PPG-5-ceteth-20, PPG-4-ceteth-20, PPG6-decyltetradeceth-30, and mixtures thereof.

Among the (poly)oxyethylenated esters, esters of polyethylene glycol and of an acid, of the following type, are in particular suitable:
R—CO—(O—CH₂—CH₂)n-OH or
R—CO—(O—CH₂—CH₂)q-O—CO—R or
R—CO—(O—CH₂—CH₂)q-O—R or
R—O—(CH(CH₃)—(CH₂))p-(O—CH₂—CH₂)q-O—CO—R
in which R, that may be identical or different, represent saturated or unsaturated, C₂-C₂₀ hydrocarbon-based groups; n, p, q, average integers, that may be identical or different, ranging from 2 to 50.

They may in particular be chosen from PEG-6 Isostearate, PEG-6 Stearate, PEG-7 cocoate, PEG-8 laurate, PEG-8 Stearate, PEG-8 Isostearate, PEG-8 oleate, PEG-9 cococate, PEG-10 oleate PEG-20 laurate, PEG-20 Stearate, PEG-30 Stearate, PEG-32 Stearate, PEG-40 laurate, PEG-40 Stearate, PEG-40 hydrogenated castor oil, PEG-75 Stearate, PEG-100 Stearate, PEG-8 Distearate, PEG-150 Distearate, Mereth-3 Myristate, PEG-4 Olivate, Propyleneglycol Ceteth-3 Acetate, PEG-30 Dipolyhydroxystearate, and also mixtures thereof.

As (poly)oxyalkylenated or non-(poly)oxyalkylenated sorbitan esters, use is preferably made of those having a number of ethylene oxide (EO) units ranging from 0 to 100, more particularly from 2 to 50 oxyethylenated units, and also polysorbates.

As regards the sorbitan esters, mention may be made of the sorbitan mono- or polyesters, preferably mono-, di- or triesters, comprising at least one saturated or unsaturated C₁₂-C₂₄ group. Mention may be made, among suitable sorbitan esters, for example, of sorbitan stearate, sorbitan isostearate, sorbitan tristearate, sorbitan laurate, sorbitan oleate, sorbitan sesquioleate, sorbitan trioleate or sorbitan palmitate, and also mixtures thereof, and preferably sorbitan stearate, sorbitan isostearate, sorbitan laurate, sorbitan oleate, sorbitan trioleate or sorbitan palmitate, and also mixtures thereof.

Among the polysorbates (INCI name), mention may be made most particularly of the compounds having the following INCI names: Polysorbate-20, Polysorbate-21, Polysorbate-60, Polysorbate-61, Polysorbate-80, Polysorbate-85, and also mixtures thereof. As regards the esters, mention may be made of PEG-40 Sorbitan Peroleate.

As regards the alkyl(poly)glucosides, use is preferably made of those containing an alkyl group including from 6 to 30 carbon atoms and preferably from 6 to 18 or even from 8 to 16 carbon atoms, and containing a glucoside group preferably comprising from 1 to 5 and notably 1, 2 or 3 glucoside units. The alkylpolyglucosides may be chosen, for example, from decylglucoside (alkyl-C₉/C₁₁-polyglucoside (1.4)), for instance the product sold under the name Mydol 10® by Kao Chemicals or the product sold under the name Plantacare 2000 UP® by Henkel and the product sold under the name Oramix NS 10® by SEPPIC; caprylyl/capryl glucoside, for instance the product sold under the name Plantacare KE 3711® by Cognis or Oramix CG 110® by SEPPIC; laurylglucoside, for instance the product sold under the name Plantacare 1200 UP® by Henkel or Plantaren 1200 N® by Henkel; cocoglucoside, for instance the product sold under the name Plantacare 818 UP® by Henkel; caprylylglucoside, for instance the product sold under the name Plantacare 810 UP® by Cognis; and mixtures thereof.

As sugar esters, mention may be made for example of glucose derivatives, such as in particular polyoxyethylenated alkylglucoses such as the compounds defined by the following INCI names: Methyl-Gluceth-10, Methyl-Gluceth-20. Also suitable are polyoxyethylenated sugar esters, for instance the compounds having the following INCI names: PEG-120 Methyl Glucose Dioleate, PEG-20 Methyl Glucose Sesquistearate, and mixtures thereof. Mention may also be made of sucrose esters, such as those chosen from sucrose cocoate, sucrose laurate, sucrose myristate, sucrose palmitate, sucrose stearate, sucrose distearate, sucrose oleate, sucrose behenate or sucrose tristearate, alone or as mixtures. Preferably, the sucrose ester is chosen from sucrose laurate, sucrose palmitate or mixtures thereof.

As (poly)glycerol ester, mention may be made of:
esters of glycerol and carboxylic acid(s) or polymer(s) of C₆-C₄₀, more particularly C₈-C₃₀, optionally hydroxylated carboxylic acid(s), or plant oil derivatives, said esters being polyoxyethylenated, comprising from 2 to 200 oxyethylene units, more preferentially from 2 to 100 oxyethylene units, even more particularly between 2 and 80 oxyethylene units. These compounds are more particularly found in the form of mono-, di- or tri-glycerides, alone or as mixtures. As examples of such esters, mention may be made of PEG-6 Caprylic/Capric Glycerides, PEG-60 Almond Glycerides, PEG-10 Olive Glycerides, PEG-45 Palm Kernel Glycerides, PEG-7 Glyceryl Cocoate, PEG-30 Glyceryl Cocoate, PEG-40 Hydrogenated Castor Oil, PEG-60 Hydrogenated Castor Oil, PEG-30 Glyceryl Stearate, PEG-200 Glyceryl Stearate, PEG-20 Glyceryl Triisostearate, PEG-70 Mango Glycerides, Hydrogenated Palm/Palm Kernel Oil PEG-6 Esters, PEG-200 Hydrogenated Glyceryl Palmate, PEG-7 Glyceryl Cocoate, the mixture of Polyoxyethylenated Palm Glycerides (200 EO) and Polyoxyethylenated Coconut Kernel Glycerides (7 EO), and also mixtures thereof.
Esters of polyglycerol comprising 2 to 20 glycerol units and of saturated or unsaturated carboxylic acid(s) or carboxylic acid polymer(s) comprising from 6 to 40 carbon atoms, preferably 8 to 30 carbon atoms, or esters of polyglycerol comprising 2 to 20 glycerol units and derivatives of plant oils, and also the mixtures thereof. Preferably, the compounds Y are chosen from non-ionic polyglycerolated esters comprising 2 to 20 glycerol units and of saturated or unsaturated carboxylic acid(s), comprising 6 to 40 carbon atoms, preferably 8 to 30 carbon atoms, or derived from plant oils, and mixtures thereof. The carboxylic acids may also comprise 1 to 3 carboxylic groups, and preferably are monocarboxylic acids. The polyglycerol compounds are more particularly mono-, di- or tri-esters. More particularly, the compounds Y are chosen from esters of polyglycerol comprising 2 to 20 glycerol units and of saturated or unsaturated carboxylic acid(s) or carboxylic acid polymer(s), comprising 6 to 40 carbon atoms, preferably 8 to 30 carbon atoms, or polyglycerol esters comprising 2 to 20 glycerol units and derived from plant oils, and also mixtures thereof. Preferably, the compounds Y are chosen from non-ionic esters of polyglycerol comprising 2 to 20 glycerol units and of saturated or unsaturated carboxylic acid(s), comprising 6 to 40 carbon atoms, preferably 8 to 30 carbon atoms, or derived from plant oils, and mixtures thereof. The carboxylic acids may also comprise 1 to 3 carboxylic groups, and preferably are monocarboxylic acids. The polyglycerol compounds are more particularly mono-, di- or tri-esters. Examples of compounds Y that may be mentioned include the following compounds, designated by their INCI name: Polyglyceryl-2 Stearate, Polyglyceryl-2 Isostearate, Polyglyceryl-2 Diisostearate, Polyglyceryl-3 Diisostearate, Polyglyceryl-3 Dicitrate/Stearate, Polyglyceryl-4 Diisostearate, Polyglyceryl-4 Caprate, Polyglyceryl-4 Laurate, Polyglyceryl-5 Laurate, Polyglyceryl-5 Oleate, Polyglyceryl-6 Caprylate, Polyglyceryl-6 Dicaprate, Polyglyceryl-6 Distearate, Polyglyceryl-6 Caprylate/Caprate, Polyglyceryl-6 Dioleate, Polyglyceryl-6 Trilaurate, Polyglyceryl-10 Laurate, Polyglyceryl-10 Dioleate, polyglycerolated apricot kernel oil esters comprising 3 to 10 glycerol units, and also mixtures thereof.

As polyoxyethylenated or non-polyoxyethylenated alkyl and polyalkyl ethers of glycerol, use is preferably made of those having a number of ethylene oxide (EO) units ranging from 0 to 100 and a number of glycerol units ranging from 1 to 30. Examples that may be mentioned include Nikkol Batyl Alcohol 100 and Nikkol Chimyl Alcohol 100.

More particularly, the surfactant(s) are chosen from sucrose esters, sorbitan esters, monoglycerolated or polyglycerolated non-ionic hydrocarbon-based surfactants and mixtures thereof.

### Anionic Surfactants

The composition according to the invention can optionally comprise at least one additional anionic, more particularly hydrocarbon-based, surfactant.

The anionic surfactants can be chosen from alkyl sulfates, alkyl ether sulfates, carboxylates, amino acid derivatives, sulfonates, isethionates, taurates, sulfosuccinates, alkylsulfoacetates, phosphates and alkyl phosphates, polypeptides or metal salts of C₁₀-C₃₀ fatty acids. More particularly, these compounds are in the form of salts of alkali metals, such as in particular sodium or potassium, or alternatively of primary or secondary, in particular C₂-C₄, amine or alkanolamine. These compounds generally comprise from 10 to 30 carbon atoms, in particular from 10 to 20 carbon atoms, in their longest hydrocarbon-based chain, and are saturated or unsaturated, and linear, branched or cyclic. They can additionally comprise up to 20 oxyalkylene units, preferably up to 15 units (in particular oxyethylene units).

### Colorant

According to one particular embodiment of the invention, the composition comprises at least one colorant, in particular chosen from synthetic and natural colorants or colorants of natural origin.

The colorant may be chosen from coated or uncoated pigments, water-soluble dyes, liposoluble dyes, and mixtures thereof.

### Pigments

The term “pigments” means white or coloured, mineral or organic particles, which are insoluble in the medium of the composition, and which are intended to colour and/or opacify the resulting composition and/or deposit.

According to one particular embodiment, the pigments used are chosen from mineral pigments.

The term “mineral pigment” refers to any pigment that satisfies the definition in Ullmann's encyclopaedia in the chapter on inorganic pigments. Among the mineral pigments that are useful in the present invention, mention may be made of zirconium oxide or cerium oxide, and also zinc oxide, iron oxide (black, yellow or red) or chromium oxide, manganese violet, ultramarine blue, chromium hydrate and ferric blue, titanium dioxide, and metal powders, for instance aluminium powder or copper powder. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

The size of the pigment that is useful in the context of the present invention is generally greater than 100 nm and may range up to 10 μm, preferably from 200 nm to 5 μm and more preferentially from 300 nm to 1 μm.

According to one particular form of the invention, the pigments have a size characterized by a D[50] of greater than 100 nm and possibly ranging up to 10 μm, preferably from 200 nm to 5 μm and more preferentially from 300 nm to 1 μm.

The sizes are measured by static light scattering using a commercial MasterSizer 3000® particle size analyzer from Malvern, which makes it possible to determine the particle size distribution of all of the particles over a wide range which may extend from 0.01 μm to 1000 μm. The data are processed on the basis of the standard Mie scattering theory. This theory is the most suitable for size distributions ranging from submicron to multimicron; it allows an “effective” particle diameter to be determined. This theory is notably described in the publication by Van de Hulst, H. C., Light Scattering by Small Particles, Chapters 9 and 10, Wiley, New York, 1957.

D[50] represents the maximum size exhibited by 50% by volume of the particles.

In the context of the present invention, the mineral pigments are more particularly iron oxide and/or titanium dioxide. Examples that may be mentioned more particularly include titanium dioxides and iron oxide coated with aluminium stearoyl glutamate, sold, for example, under the reference NAI® by Miyoshi Kasei.

As mineral pigments that may be used in the invention, mention may also be made of pearlescent agents.

The term “pearlescent agents” should be understood as meaning coloured particles of any form, which may or may not be iridescent, notably produced by certain molluscs in their shell, or alternatively synthesized, and which have a colour effect via optical interference.

The pearlescent agents may be chosen from pearlescent pigments such as titanium mica coated with an iron oxide, titanium mica coated with bismuth oxychloride, titanium mica coated with chromium oxide, titanium mica coated with an organic dye and also pearlescent pigments based on bismuth oxychloride. They may also be mica particles, at the surface of which are superposed at least two successive layers of metal oxides and/or of organic colorants.

Examples of pearlescent agents that may also be mentioned include natural mica covered with titanium oxide, with iron oxide, with natural pigment or with bismuth oxychloride.

The pearlescent agents can more particularly have a yellow, pink, red, bronze, orangey, brown, gold and/or coppery colour or tint.

Among the pigments that may be used according to the invention, mention may also be made of those having an optical effect different from a simple conventional colouring effect, i.e. a unified and stabilized effect such as produced by conventional colorants, for instance monochromatic pigments. For the purposes of the invention, the term “stabilized” means lacking the effect of variability of the colour with the angle of observation or in response to a temperature change.

For example, this material may be chosen from particles with a metallic tint, goniochromatic colouring agents, diffractive pigments, thermochromic agents, optical brighteners, and also fibres, notably interference fibres. Needless to say, these various materials may be combined in order simultaneously to afford two effects, or even a novel effect in accordance with the invention.

According to one particular embodiment, the composition according to the invention comprises at least one uncoated pigment.

According to another particular embodiment, the composition according to the invention comprises at least one pigment coated by at least one lipophilic or hydrophobic compound.

This type of pigment is particularly advantageous. Insofar as they are treated with a hydrophobic compound, they show predominant affinity for an oily phase, which can then convey them.

The coating may also comprise at least one additional non-lipophilic compound.

For the purposes of the invention, the “coating” of a pigment according to the invention generally denotes the total or partial surface treatment of the pigment with a surface agent, absorbed, adsorbed or grafted onto said pigment.

The surface-treated pigments may be prepared according to surface treatment techniques of chemical, electronic, mechanochemical or mechanical nature that are well known to those skilled in the art. Commercial products may also be used.

The surface agent may be absorbed, adsorbed or grafted onto the pigments by evaporation of solvent, chemical reaction and creation of a covalent bond.

According to one variant, the surface treatment consists in coating the pigments.

The coating may represent from 0.1% to 20% by weight and in particular from 0.5% to 5% by weight relative to the total weight of the coated pigment.

The coating may be produced, for example, by adsorption of a liquid surface agent onto the surface of the solid particles by simple mixing with stirring of the particles and of said surface agent, optionally with heating, prior to the incorporation of the particles into the other ingredients of the makeup or care composition.

The coating may be produced, for example, by chemical reaction of a surface agent with the surface of the solid pigment particles and creation of a covalent bond between the surface agent and the particles. This method is notably described in U.S. Pat. No. 4,578,266.

The chemical surface treatment may consist in diluting the surface agent in a volatile solvent, dispersing the pigments in this mixture and then slowly evaporating off the volatile solvent, so that the surface agent is deposited on the surface of the pigments.

When the pigment comprises a lipophilic or hydrophobic coating, it is preferably present in the fatty phase of the composition according to the invention.

According to one particular embodiment of the invention, the pigments may be coated according to the invention with at least one compound chosen from silicone surface agents; fluoro surface agents; fluorosilicone surface agents; metal soaps; N-acylamino acids or salts thereof; lecithin and derivatives thereof; isopropyl triisostearyl titanate; isostearyl sebacate; natural plant or animal waxes; polar synthetic waxes; fatty esters; phospholipids; and mixtures thereof.

According to one particular embodiment of the invention, the pigments may be coated with a hydrophilic compound.

According to another particular embodiment, the colorant is an organic pigment, which is synthetic, natural or of natural origin.

The term “organic pigment” refers to any pigment that satisfies the definition in Ullmann's Encyclopedia in the chapter on organic pigments. The organic pigment may notably be chosen from nitroso, nitro, azo, xanthene, quinoline, anthraquinone, phthalocyanine, metal-complex type, isoindolinone, isoindoline, quinacridone, perinone, perylene, diketopyrrolopyrrole, thioindigo, dioxazine, triphenylmethane or quinophthalone compounds.

The organic pigment(s) may be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as described in patent FR 2 679 771.

The pigments may also be in the form of composite pigments as described in patent EP 1 184 426. These composite pigments may notably be composed of particles including a mineral core at least partially covered with an organic pigment and at least one binder for fixing the organic pigments to the core.

The pigment may also be a lake. The term “lake” means insolubilized dyes adsorbed onto insoluble particles, the assembly thus obtained remaining insoluble during use.

The mineral substrates onto which the dyes are adsorbed are, for example, alumina, silica, calcium sodium borosilicate or calcium aluminium borosilicate and aluminium.

Among the organic dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

An example of a lake that may be mentioned is the product known under the name D&C Red 7 (CI 15 850:1).

The pigment(s) are preferably present in the composition according to the invention in contents of at least 0.01% by weight, more particularly of at least 1% by weight, and even more particularly of at least 2% by weight, relative to the weight of the composition concerned. More particularly, the colorant content is less than 50% by weight, more particularly between 0.05% and 30% by weight, and better still from 0.1% to 25% by weight, relative to the total weight of the composition.

### Water-Soluble or Liposoluble Dyes

According to one particular embodiment of the invention, the colorant is a water-soluble dye or a liposoluble dye.

For the purposes of the invention, the term “water-soluble colorant” means any natural or synthetic, generally organic compound, which is soluble in an aqueous phase or water-miscible solvents and which is capable of colouring.

For the purposes of the invention, the term “liposoluble colorant” means any natural or synthetic, generally organic compound, which is soluble in an oily phase or in solvents that are miscible with the oily phase, and which is capable of imparting colour.

As water-soluble dyes that are suitable for use in the invention, mention may notably be made of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5 and FDC Blue 1.

Among the natural water-soluble dyes, mention may be made of anthocyanins.

As liposoluble dyes that are suitable for use in the invention, mention may notably be made, for instance, of the liposoluble dyes DC Red 17, DC Red 21, DC Red 27, DC Green 6, DC Yellow 11, DC Violet 2, DC Orange 5, Sudan red and Sudan brown.

As illustrations of natural liposoluble dyes, mention may be made particularly of carotenes, for instance β-carotene, α-carotene and lycopene; quinoline yellow; xanthophylls such as astaxanthin, antheraxanthin, citranaxanthin, cryptoxanthin, canthaxanthin, diatomoxanthin, flavoxanthin, fucoxanthin, lutein, rhodoxanthin, rubixanthin, siphonaxanthin, violaxanthin, zeaxanthin; annatto; curcumin; quinizarin (Ceres Green BB, D&C Green No. 6, CI 61565, 1,4-di-p-toluidinoanthraquinone, Green No. 202, quinazine green SS) and chlorophylls.

The water-soluble or liposoluble dye(s) are preferably present in the composition according to the invention in contents of less than 4% by weight, or even less than 2% by weight, more preferentially ranging from 0.01% to 2% by weight and even better still from 0.02% to 1.5% by weight, relative to the total weight of the composition.

### Thickeners

The composition contest invention may comprise at least one aqueous-phase thickener and/or at least one lipophilic-phase thickener.

### Aqueous-Phase Thickeners

As examples of aqueous-phase thickening polymers, mention may be made more particularly of:
acrylic or methacrylic acid homopolymers or copolymers or salts and esters thereof and in particular the products sold under the names Versicol F or Versicol K by Allied Colloid, Ultrahold 8 by BASF, and polyacrylic acids of Synthalen K type, and salts, in particular sodium salts, of polyacrylic acid (corresponding to the INCI name sodium acrylate copolymer) and more particularly a crosslinked sodium polyacrylate (corresponding to the INCI name sodium acrylate copolymer (and) caprylic/capric triglyceride) sold under the name Luvigel EM by the BASF,
copolymers of acrylic acid and of acrylamide sold in the form of the sodium salt thereof under the Reten names by Hercules, the sodium polymethacrylate sold under the name Darvan No. 7 by Vanderbilt, and the sodium salts of polyhydroxycarboxylic acids sold under the name Hydagen F by Henkel,
polyacrylic acid/alkyl acrylate copolymers, preferably modified or unmodified carboxyvinyl polymers, particularly with acrylate/C₁₀-C₃₀-alkylacrylate copolymers (INCI name: Acrylates/C₁₀-C₃₀ Alkyl acrylate Crosspolymer) such as the products sold by Lubrizol under the trade names Pemulen TR1, Pemulen TR2, Carbopol 1382 and Carbopol EDT 2020, and even more preferentially Pemulen TR-2,
polyacrylamidomethylpropanesulfonic acid partially neutralized with aqueous ammonia and highly crosslinked, sold by Clariant,
acrylamidopropanesulfonic/acrylamide copolymers of Sepigel or Simulgel type sold by SEPPIC,
polyoxyethylenated acrylamidopropanesulfonic/alkyl methacrylate copolymers (crosslinked or non-crosslinked) of the Aristoflex HMS type sold by Clariant,
copolymers of hydroxyalkylacrylic acid or salts thereof and of acryloyldimethyl taurate monomers such as the Sepinov EMT 10 products sold by SEPPIC,
and mixtures thereof.

Other examples of aqueous-phase thickening polymers that may be mentioned include:
anionic, cationic, amphoteric or non-ionic chitin or chitosan polymers;
cellulose polymers, for instance alkylcelluloses such as hydroxyethylcellulose, hydroxypropylcellulose, hydroxymethylcellulose, ethylhydroxyethylcellulose and carboxymethylcellulose, and also quaternized cellulose derivatives;
vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;
optionally modified polymers of natural origin, such as galactomannans and derivatives thereof, for instance konjac gum, gellan gum, locust bean gum, fenugreek gum, karaya gum, gum tragacanth, gum arabic, acacia gum, guar gum, hydroxypropyl guar, hydroxypropyl guar modified with sodium methylcarboxylate groups (Jaguar XC97-1, Rhodia), hydroxypropyltrimethylammonium guar chloride, and xanthan gum and derivatives thereof;
alginates and carrageenans;
and mixtures thereof.

If the composition comprises any, the content of aqueous-phase thickener ranges from 0.01% to 3% by weight, preferably from 0.05% to 2% by weight and more advantageously from 0.1% to 1% by weight relative to the total weight of the composition.

### Hydrophobic-Phase Thickeners

By way of hydrophobic-phase thickeners, mention may most particularly be made of hydrophobic mineral thickeners such as modified clays, modified silicas, or mixtures thereof.

### Hydrophobically Modified Clays

Clays are silicates containing a cation that may be chosen from calcium, magnesium, aluminium, sodium, potassium and lithium cations, and mixtures thereof.

Mention may be made, as examples of such products, of clays of the family of the smectites, and also of the family of the vermiculites, stevensite or chlorites. These clays may be of natural or synthetic origin.

Preferably, use is made of organophilic clays, more particularly of modified clays, such as montmorillonite, bentonite, hectorite, attapulgite or sepiolite, and mixtures thereof. The clay is preferably a bentonite or a hectorite.

These clays are modified with a chemical compound chosen from quaternary amines, tertiary amines, amine acetates, imidazolines, amine soaps, fatty sulfates, alkylarylsulfonates or amine oxides, and mixtures thereof.

Mention may thus be made of hectorites modified by a quaternary amine, more specifically by a C₁₀ to C₂₂ fatty acid ammonium halide, such as chloride, comprising or not comprising an aromatic group, such as hectorite modified by a distearyldimethylammonium halide, preferably chloride (CTFA name: Disteardimonium hectorite), such as, for example, that sold under the name Bentone 38V, Bentone 38V CG or Bentone EW CE by Elementis, or stearalkonium hectorites, such as in particular the product Bentone 27 V.

Mention may also be made of quaternium-18 bentonites, such as those sold, inter alia, under the names Bentone 34 by Elementis, Claytone 40, Tixogel VP by United Catalyst by Southern Clay; stearalkonium bentonites, such as those sold under the names Tixogel LG by United Catalyst and Claytone AF and Claytone APA by Southern Clay; or quaternium-18/benzalkonium bentonites, such as those sold under the name Claytone HT by Southern Clay.

According to one preferred embodiment, the thickening agent is chosen from organophilic modified clays, in particular organophilic modified hectorites, in particular modified by benzyldimethylammonium stearate or distearyldimethylammonium halides, in particular chlorides.

### Modified Silicas

Mention may also be made of fumed silica hydrophobically treated at the surface, the size of the particles of which is advantageously less than 1 μm. It is in fact possible to chemically modify the surface of the silica, by chemical reaction generating a reduction in the number of silanol groups present at the surface of the silica. It is notably possible to substitute silanol groups with hydrophobic groups: a hydrophobic silica is then obtained. The hydrophobic groups may be:
trimethylsiloxyl groups, which are notably obtained by treating fumed silica in the presence of hexamethyldisilazane. Silicas thus treated are known as Silica Silylate according to the CTFA (6th edition, 1995). They are sold, for example, under the references Aerosil R812® by Degussa, and Cab-O-Sil TS-530® by Cabot,
dimethylsilyloxyl or polydimethylsiloxane groups, which are obtained especially by treating fumed silica in the presence of polydimethylsiloxane, for example hexamethyldisiloxane, or dimethyldichlorosilane. Silicas thus treated are known as Silica Dimethyl Silylate according to the CTFA (6th edition, 1995). They are sold, for example, under the references Aerosil R972® and Aerosil R974® by Degussa, and Cab-O-Sil TS-610® and Cab-O-Sil TS-720® by Cabot.

The hydrophobic fumed silica in particular has a particle size that may be nanometric to micrometric, for example ranging from about from 5 to 200 nm.

The composition according to the invention can also comprise at least silica aerogel particles.

Silica aerogels are porous materials obtained by replacing (by drying) the liquid component of a silica gel with air.

They are generally synthesized via a sol-gel process in a liquid medium and then dried, usually by extraction with a supercritical fluid, the one most commonly used being supercritical CO₂. This type of drying makes it possible to avoid shrinkage of the pores and of the material. The sol-gel process and the various drying operations are described in detail in Brinker C. J. and Scherer G. W., Sol-Gel Science, New York, Academic Press, 1990.

The hydrophobic silica aerogel particles suitable for the implementation of the invention exhibit a specific surface area per unit mass (S_{M}) ranging from 500 to 1500 m²/g, preferably from 600 to 1200 m²/g and better still from 600 to 800 m²/g, and a size, expressed as the volume-average diameter (D[0.5]), ranging from 1 to 1500 μm, better still from 1 to 1000 μm, preferably from 1 to 100 μm, in particular from 1 to 30 μm, more preferably from 5 to 25 μm, better still from 5 to 20 μm and even better still from 5 to 15 μm.

According to one advantageous embodiment, the hydrophobic silica aerogel particles used in the present invention have a specific surface area per unit mass (S_{M}) ranging from 600 to 800 m²/g and a size expressed as the volume mean diameter (D[0.5]) ranging from 5 to 20 μm and even better still from 5 to 15 μm.

The specific surface per unit of weight can be determined by the nitrogen absorption method, known as the BET (Brunauer-Emmett-Teller) method, described in The Journal of the American Chemical Society, Vol. 60, page 309, February 1938 and corresponding to the international standard ISO 5794/1 (appendix D). The BET specific surface area corresponds to the total specific surface area of the particles under consideration.

The sizes of the silica aerogel particles may be measured by static light scattering using a commercial particle size analyzer such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is in particular described in the publication by Van de Hulst, H. C., Light Scattering by Small Particles, Chapters 9 and 10, Wiley, New York, 1957.

According to one preferred embodiment, the hydrophobic silica aerogel particles used in the present invention have a specific surface area per unit of volume Sᵥ ranging from 5 to 60 m²/cm³, preferably from 10 to 50 m²/cm³ and better still from 15 to 40 m²/cm³.

The aerogels used according to the present invention are hydrophobic silica aerogels, preferably aerogels of silyl silica (INCI name: Silica Silylate).

As regards the preparation of hydrophobic silica aerogel particles that have been surface-modified by silylation, reference may be made to U.S. Pat. No. 7,470,725.

Use will preferably be made of hydrophobic silica aerogel particles surface-modified with trimethylsilyl groups.

As hydrophobic silica aerogels that may be used in the invention, examples that may be mentioned include the aerogel sold under the name VM-2260 (INCI name: Silica silylate), by Dow Corning, the particles of which have an average size of about 1000 microns and a specific surface area per unit mass ranging from 600 to 800 m²/g.

Mention may also be made of the aerogels sold by Cabot under the references Aerogel TLD 201, Aerogel OGD 201, Aerogel TLD 203, Enova® Aerogel MT 1100 and Enova Aerogel MT 1200.

Use will preferably be made of the aerogel sold under the name VM-2270 (INCI name: Silica silylate) by Dow Corning, the particles of which have an average size ranging from 5-15 microns and a specific surface area per unit of mass ranging from 600 to 800 m²/g.

Preferably, the mineral thickeners are chosen from organophilic clays, in particular modified hectorites; hydrophobic treated fumed silica; hydrophobic silica aerogels, or mixtures thereof, and more specifically still at least one organophilic modified clay or at least one hydrophobically modified silica, in particular an organophilic modified clay.

More particularly, if the composition contains it, the content of hydrophobic-phase thickener(s) represents from 0.2% to 2.5% by weight, expressed as active material, and preferably from 0.5% to 2% by weight, relative to the total weight of the composition.

Throughout the description, including the claims, the expression “comprising a” should be understood as being synonymous with “comprising at least one”, unless otherwise specified.

The expressions “between . . . and . . . ” and “ranging from . . . to . . . ” should be understood as meaning limits included, unless otherwise specified.

In addition, the sum of the amounts of the ingredients of the composition represents 100% by weight of the composition.

The invention is illustrated in greater detail by the examples presented below.

Unless otherwise indicated, the amounts indicated are expressed as percentages by weight of starting materials.

The starting materials are referred to by their chemical name or their INCI name.

### EXAMPLES

### Example 1

In the following table, the amount of each compound is given as a weight percentage/weight of composition (the percentages are expressed as weight of starting material unless otherwise indicated).

TABLE 1

| | Comparative | |
|---|---|---|
| Ingredients (INCI name, chemical name) | composition A | Composition 1 |
| Hydrogenated Coco-Glycerides | 27 | 27 |
| (Softisan 100 - Cremer Oleo) | | |
| Ethylcellulose | 47.63 | 40 |
| (aqueous dispersion with 26.2% of ethylcellulose; | | |
| Aquacoat ECD-30 - FMC Biopolymer) | | |
| Octyldodecanol | 4 | 4 |
| Dehydroxanthan Gum | 0.15 | 0.15 |
| (Amaze XT - Akzo Nobel) | | |
| Euphorbia Cerifera (Candelilla) Wax Extract | — | 2 |
| (Candellila Resin E-1 - Japan Natural Products) | | |
| Tridecyl Stearate, Neopentyl Glycol Dicaprylate/ | 20.12 | 20.85 |
| Dicaprate, Tridecyl Trimellitate (Lipovol MOS - | | |
| 70 - Vantage Specialty Chemicals) | | |
| Cetyl alcohol | 1.00 | 1.00 |
| Water | — | 4.90 |
| Red 33 | 0.10 | 0.10 |
| % Ethylcellulose and candelilla resin | 12.5% | 12.5% |

### Procedure

The following steps are carried out:
1. The Softisan 100 and the octyldodecanol are mixed at 60° C. and then this mixture is poured into the aqueous dispersion of ethylcellulose, with stirring using the Rayneri deflocculator for 10 minutes at 1200 rpm.
2. The xanthan is added and the stirring is continued for 30 minutes.
3. The dyes solubilized in water (Composition 1) are then introduced or are dusted onto the mixture (Composition A) and stirring is carried out for 15 minutes.
4. Furthermore, the liquid esters (Lipovol MOS-70) and the candelilla resin are mixed and the resulting mixture is heated at 85° C. Once the mixture is homogeneous, the cetyl alcohol is added thereto with stirring.
5. When the preparations homogeneous, it is cooled to 55° C. and is poured onto the mixture obtained at the outcome of step 3. The whole mixture is emulsified for 15 minutes and then left to cool to ambient temperature with stirring (1200 rpm). The mixture is then packaged in a gloss bottle (pot fitted with a flocked applicator—reference 14030, Geka GmbH).

### Protocols for Evaluating the Wear Property and the Transfer Resistance:

### 1. Preparation of the Samples

An area 2 cm×2 cm on the inner side of the forearm is made up with the composition by applying 4 layers without re-immersing the applicator in the pot. This operation is reproduced so as to have four made-up samples (a control sample and 3 samples to be tested: water, oil, transfer).

The product is left to dry for 30 minutes.

2. The water resistance is evaluated by passing cotton wool, soaked in 1 ml of water, over the surface of the first sample, once. The colour of the deposit thus treated and that of the control deposit are compared.

3. The oil resistance is evaluated by passing cotton wool, soaked in 0.5 ml olive oil (similar applied force to that of the water resistance) over the surface of the second sample, once. The colour of the deposit thus treated and that of the control deposit are compared.

4. The transfer resistance is evaluated by pressing a paper tissue on the third sample and by observing the amount of lipstick deposited on the tissue.

### Evaluation of the Compositions:

A stable, homogeneous liquid composition is obtained in each case.

The compositions apply easily to the lips, as a fine and fresh deposit which is only very slightly tacky and which does not migrate.

It is noted that the coverage and the intensity of the colour are greater in the case of the composition according to the invention.

The compositions do not transfer.

However, it is noted that the water resistance and oil resistance of the composition according to the invention are significantly improved compared to those of the comparative composition.

### Example 2

In the following table, the amount of each compound is given as a weight percentage/weight of composition (the percentages are expressed as weight of starting material unless otherwise indicated).

TABLE 2

| | Composition B | |
|---|---|---|
| Ingredients (INCI name, chemical name) | comparative | Composition 2 |
| Hydrogenated Coco-Glycerides | 27 | 27 |
| (Softisan 100 - Cremer Oleo) | | |
| Ethylcellulose | 40 | 40 |
| (aqueous dispersion with 26.2% of ethylcellulose; | | |
| Aquacoat ECD-30 - FMC Biopolymer) | | |
| Octyldodecanol | 4 | 4 |
| Dehydroxanthan Gum | 0.15 | 0.15 |
| (Amaze XT - Akzo Nobel) | | |
| Euphorbia Cerifera (Candelilla) Wax Extract | — | 2 |
| (Candellila Resin E-1 - Japan Natural Products) | | |
| Myristoyl Pullulan (Katakura Chikkarin) | 2 | — |
| Neopentyl Glycol Dicaprate | 20.85 | 20.85 |
| (Estemol N-01- Nisshin Oillio) | | |
| Cetyl alcohol | 1.00 | 1.00 |
| Water | 4.90 | 4.90 |
| Red 33 | 0.10 | 0.10 |

The following steps are carried out:
1. The Softisan 100 and the octyldodecanol are mixed at 60° C. and then this mixture is poured into the aqueous dispersion of ethylcellulose, with stirring using the Rayneri deflocculator for 10 minutes at 1200 rpm.
2. The xanthan is added and the stirring is continued for 30 minutes.
3. The dyes solubilized in water are then introduced and stirring is carried out for 15 minutes.
4. Furthermore, the neopentyl dicaprate and the candelilla resin or the myristoyl pullulan are mixed and the resulting mixture is heated at 85° C. Once the mixture is homogeneous, the cetyl alcohol is added thereto with stirring.
5. When the preparations homogeneous, it is cooled to 55° C. and is poured onto the mixture obtained at the outcome of step 3. The whole mixture is emulsified for 15 minutes and then left to cool to ambient temperature with stirring. The mixture is then packaged in a gloss bottle (pot fitted with a flocked applicator—reference 14030, Geka GmbH).

### Evaluation of the Compositions

The compositions obtained are liquid, homogeneous and stable.

Each composition applies easily to the lips, as a fine and fresh deposit which is only very slightly tacky and which does not migrate. The deposits are intense and covering.

The composition according to the invention does not transfer, unlike the comparative composition (protocol defined in Example 1).

It is also noted that the wear property of the composition according to the invention is significantly improved relative to water and to oil (protocols defined in Example 1).

### Example 3

In the following table, the amount of each compound is given as a weight percentage/weight of composition (the percentages are expressed as weight of starting material unless otherwise indicated).

TABLE 3

| Ingredients (INCI name, chemical name) | Composition 3 | Composition 4 |
|---|---|---|
| Hydrogenated Coco-Glycerides | 29 | 29 |
| (Softisan 100 - Cremer Oleo) | | |
| Ethylcellulose | 36 | 40 |
| (aqueous dispersion with 26.2% of ethylcellulose; | | |
| Aquacoat ECD-30 - FMC Biopolymer) | | |
| Octyldodecanol | 10.5 | 12.5 |
| VP/Eicosene Copolymer | 4.5 | 4.5 |
| (Antaron V 220F polymer - ISP) | | |
| Euphorbia Cerifera (Candelilla) Wax Extract | — | 9 |
| (Candellila Resin E-1 - Japan Natural Products) | | |
| Shorea Robusta Resin | 15 | — |
| (as a mixture in octyldodecanol; Kahlresine 6723 - | | |
| Kahl) | | |
| Neopentyl Glycol Dicaprate | 20.85 | 20.85 |
| (Estemol N-01- Nisshin Oillio) | | |
| Cetyl alcohol | 1.00 | 1.00 |
| Water | 4.90 | 4.90 |
| Red 33 | 0.10 | 0.10 |

### Procedure

The following steps are carried out:
1. The octyldodecanol and the candelilla resin or the Shorea resin are mixed at 85° C. with stirring. Once the mixture is homogeneous, the VP/Eicosene copolymer and then the Softisan 100 are added while maintaining the stirring. Once the mixture is homogenized, it is cooled to 55° C. and is then added to the dispersion of ethylcellulose.
2. The dyes solubilized in water are then introduced and stirring is carried out for 15 minutes.
3. Furthermore, the neopentyl dicaprate and the candelilla resin or the Shorea robusta resin are mixed and the resulting mixture is heated at 85° C. Once the mixture is homogeneous, the cetyl alcohol is added thereto with stirring.
4. When the preparations homogeneous, it is cooled to 55° C. and is poured onto the mixture obtained at the outcome of step 3. The whole mixture is emulsified for 15 minutes and then left to cool to ambient temperature with stirring. The mixture is then packaged in a gloss bottle (pot fitted with a flocked applicator—reference 14030, Geka GmbH).

### Evaluation of the Compositions

The resulting compositions are liquid, homogeneous and stable.

They apply easily to the lips. The deposits are fine, with a feeling of freshness on application, are only very slightly tacky, and do not migrate.

The deposits do not transfer and they exhibit very good water resistance and oil resistance (protocols defined in Example 1).

### Example 4

In the following table, the amount of each compound is given as a weight percentage/weight of composition (the percentages are expressed as weight of starting material unless otherwise indicated).

TABLE 4

| | Comparative | Comparative | |
|---|---|---|---|
| Ingredients (INCI name, chemical name) | Composition C | composition D | Composition 5 |
| Hydrogenated Coco-Glycerides | 29 | 29 | 29 |
| (Softisan 100 - Cremer Oleo) | | | |
| Ethylcellulose | 40 | — | 24.7 |
| (aqueous dispersion; 26.2% of ethyl- | | | |
| cellulose; Aquacoat ECD-30 - FMC | | | |
| Biopolymer) | | | |
| Octyldodecanol | 21.5 | 12.5 | 17.6 |
| Polysorbate 80 | — | 2 | — |
| VP/Eicosene Copolymer | 4.5 | 4.5 | 4.5 |
| (Antaron V 220F polymer - ISP) | | | |
| Euphorbia Cerifera (Candelilla) Wax | — | 10.48 | 4 |
| Extract | | | |
| (Candellila Resin E-1 - Japan Natural | | | |
| Products) | | | |
| Water | 4.90 | 38.42 | 20.10 |
| Red 33 | 0.10 | 0.10 | 0.10 |

### Procedure

The following steps are carried out:
1. The octyldodecanol and the candelilla resin are mixed at 85° C. with stirring. Once the mixture is homogeneous, the Softisan 100 and then the VP/Eicosene copolymer are added while maintaining the stirring. Once the mixture is homogenized, it is cooled to 55° C. and is then added to the dispersion of ethylcellulose heated to 55° C.
3. The polysorbate 80 is added, then the whole mixture is emulsified for 15 minutes and then left to cool to ambient temperature with stirring.
4. The dyes solubilized in water are then introduced and stirring is carried out for 15 minutes. The mixture is then packaged in a gloss bottle (pot fitted with a flocked applicator—reference 14030, Geka GmbH).

### Evaluation of the Compositions

Compositions C and 5 are liquid, homogeneous and stable compositions, whereas phase separation occurs in Composition D and said composition cannot be applied

Compositions C and 5 apply easily to the lips, as a fine and fresh deposit which is only very slightly tacky and which does not migrate. The deposits do not transfer (protocol defined in Example 1).

Composition 5 according to the invention exhibits water resistance and oil resistance that are significantly improved compared to Comparative Composition C (protocol defined in Example 1).

### Example 5

In the following table, the amount of each compound is given as a weight percentage/weight of composition (the percentages are expressed as weight of starting material unless otherwise indicated).

TABLE 5

| Ingredients (INCI name, chemical name) | Composition 6 |
|---|---|
| Hydrogenated Coco-Glycerides | 27 |
| (Softisan 100 - Cremer Oleo) | |
| Ethylcellulose | 42.73 |
| (aqueous dispersion with 26.2% of ethylcellulose; | |
| Aquacoat ECD-30 - FMC Biopolymer) | |
| Octyldodecanol | 4 |
| Dehydroxanthan Gum | 0.15 |
| (Amaze XT - Akzo Nobel) | |
| Euphorbia Cerifera (Candelilla) Wax Extract | 2 |
| (Candellila Resin E-1 - Japan Natural Products) | |
| Neopentyl Glycol Dicaprate | 18.12 |
| (Estemol N-01- Nisshin Oillio) | |
| Cetyl alcohol | 1.00 |
| Water | 4.90 |
| Red 33 | 0.10 |

### Procedure

The following steps are carried out:
1. The Softisan 100 and the octyldodecanol are mixed at 60° C. and then this mixture is poured into the aqueous dispersion of ethylcellulose, with stirring using the Rayneri deflocculator for 10 minutes at 1200 rpm.
2. The xanthan is added and the stirring is continued for 30 minutes.
3. The dyes solubilized in water are then introduced and stirring is carried out for 15 minutes.
4. Furthermore, the neopentyl dicaprate and the candelilla resin are mixed and the resulting mixture is heated at 85° C. Once the mixture is homogeneous, the cetyl alcohol is added thereto with stirring.
5. When the preparations homogeneous, it is cooled to 55° C. and is poured onto the mixture obtained at the outcome of step 3. The whole mixture is emulsified for 15 minutes and then left to cool to ambient temperature with stirring. The mixture is then packaged in a gloss bottle (pot fitted with a flocked applicator—reference 14030, Geka GmbH).

### Evaluation of the Composition

A stable, homogeneous liquid composition is obtained.

The composition applies easily to the lips, as a fine and fresh deposit which is only very slightly tacky, which does not migrate and which does not transfer (protocol of Example 1).

It is noted that the coverage, the colour intensity and the resistance to water and oil are very good (protocols of Example 1).

### Example 6

In the following table, the amount of each compound is given as a weight percentage/weight of composition (the percentages are expressed as weight of starting material unless otherwise indicated).

TABLE 6

| Ingredients (INCI name, chemical name) | Composition 7 |
|---|---|
| Hydrogenated Coco-Glycerides | 27 |
| (Softisan 100 - Cremer Oleo) | |
| Ethylcellulose | 40 |
| (aqueous dispersion; 26.2% of ethylcellulose; | |
| Aquacoat ECD-30 - FMC Biopolymer) | |
| Octyldodecanol | 4 |
| Gellan Gum | 0.15 |
| (Kelcogel CG LA- CP Kelco) | |
| Euphorbia Cerifera (Candelilla) Wax Extract | 2 |
| (Candellila Resin E-1 - Japan Natural Products) | |
| Neopentyl Glycol Dicaprate | 18.12 |
| (Estemol N-01- Nisshin Oillio) | |
| Cetyl alcohol | 1.00 |
| Water | 4.90 |
| Red 33 | 0.10 |

### Procedure

The following steps are carried out:
1. The Softisan 100 and the octyldodecanol are mixed at 60° C. and then this mixture is poured into the aqueous dispersion of ethylcellulose, with stirring using the Rayneri deflocculator for 10 minutes at 1200 rpm.
2. The gellan gum is added and the stirring is continued for 30 minutes.
3. The dyes solubilized in water are then introduced and stirring is carried out for 15 minutes.
4. Furthermore, the neopentyl dicaprate and the candelilla resin are mixed and the resulting mixture is heated at 85° C. Once the mixture is homogeneous, the cetyl alcohol is added thereto with stirring.
5. When the preparations homogeneous, it is cooled to 55° C. and is poured onto the mixture obtained at the outcome of step 3. The whole mixture is emulsified for 15 minutes and then left to cool to ambient temperature with stirring. The mixture is then packaged in a gloss bottle (pot fitted with a flocked applicator—reference 14030, Geka GmbH).

### Evaluation of the Composition

A stable, homogeneous liquid composition is obtained.

The composition applies easily to the lips, as a fine and fresh deposit which is only very slightly tacky, which does not migrate and which does not transfer (protocol of Example 1).

It is noted that the coverage, the colour intensity and the resistance to water and oil are very good (protocols of Example 1).

### Example 7

In the following table, the amount of each compound is given as a weight percentage/weight of composition (the percentages are expressed as weight of starting material unless otherwise indicated).

TABLE 7

| Ingredients (INCI name, chemical name) | Composition 8 |
|---|---|
| Hydrogenated Coco-Glycerides | 27 |
| (Softisan 100 - Cremer Oleo) | |
| Ethylcellulose | 40 |
| (aqueous dispersion; 26.2% of ethylcellulose; | |
| Aquacoat ECD-30 - FMC Biopolymer) | |
| Octyldodecanol | 4 |
| Gellan Gum | 0.15 |
| (Kelcogel CG LA- CP Kelco) | |
| Euphorbia Cerifera (Candelilla) Wax Extract | 2 |
| (Candellila Resin E-1 - Japan Natural Products) | |
| Dicaprylyl ether | 18.12 |
| (Cetiol OE - BASF) | |
| Cetyl alcohol | 1.00 |
| Water | 4.90 |
| Red 33 | 0.10 |

### Procedure

The following steps are carried out:
1. The Softisan 100 and the octyldodecanol are mixed at 60° C. and then this mixture is poured into the aqueous dispersion of ethylcellulose, with stirring using the Rayneri deflocculator for 10 minutes at 1200 rpm.
2. The gellan gum is added and the stirring is continued for 30 minutes.
3. The dyes solubilized in water are then introduced and stirring is carried out for 15 minutes.
4. Furthermore, the dicaprylyl ether and the candelilla resin are mixed and the resulting mixture is heated at 85° C. Once the mixture is homogeneous, the cetyl alcohol is added thereto with stirring.
5. When the preparations homogeneous, it is cooled to 55° C. and is poured onto the mixture obtained at the outcome of step 3. The whole mixture is emulsified for 15 minutes and then left to cool to ambient temperature with stirring. The mixture is then packaged in a gloss bottle (pot fitted with a flocked applicator—reference 14030, Geka GmbH).

### Evaluation of the Composition

A stable, homogeneous liquid composition is obtained.

The composition applies easily to the lips, as a fine and fresh deposit which is which is only very slightly tacky, which does not migrate and which does not transfer (protocol of Example 1).

It is noted that the coverage, the colour intensity and the resistance to water and oil are very good (protocols of Example 1).

### Comparative Example 8

In the following table, the amount of each compound is given as a weight percentage/weight of composition (the percentages are expressed as weight of starting material unless otherwise indicated).

TABLE 8

| Ingredients (INCI name, chemical name) | Composition E |
|---|---|
| VP/Eicosene Copolymer | 27 |
| (Antaron V 220F polymer - ISP) | |
| Ethylcellulose | 40 |
| (aqueous dispersion; 26.2% of ethylcellulose; | |
| Aquacoat ECD-30 - FMC Biopolymer) | |
| Octyldodecanol | 4 |
| Gellan Gum | 0.15 |
| (Kelcogel CG LA- CP Kelco) | |
| Euphorbia Cerifera (Candelilla) Wax Extract | 2 |
| (Candellila Resin E-1 - Japan Natural Products) | |
| Neopentyl Glycol Dicaprate | 18.12 |
| (Estemol N-01- Nisshin Oillio) | |
| Cetyl alcohol | 1.00 |
| Water | 4.90 |
| Red 33 | 0.10 |

### Procedure

The following steps are carried out:
1. The VP/Eicosene copolymer and the octyldodecanol are mixed at 60° C. and then this mixture is poured into the aqueous dispersion of ethylcellulose, with stirring using the Rayneri deflocculator for 10 minutes at 1200 rpm.
2. The gellan gum is added and the stirring is continued for 30 minutes.
3. The dyes solubilized in water are then introduced and stirring is carried out for 15 minutes.
4. Furthermore, the neopentyl glycol and the candelilla resin are mixed and the resulting mixture is heated at 85° C. Once the mixture is homogeneous, the cetyl alcohol is added thereto with stirring.
5. When the preparations homogeneous, it is cooled to 55° C. and is poured onto the mixture obtained at the outcome of step 3. The whole mixture is emulsified for 15 minutes and then left to cool to ambient temperature with stirring. The mixture is then packaged in a gloss bottle (pot fitted with a flocked applicator—reference 14030, Geka GmbH).

### Evaluation of the Composition

A composition which is homogeneous but not too thick to be applied uniformly is obtained.

### Example 9

In the following table, the amount of each compound is given as a weight percentage/weight of composition (the percentages are expressed as weight of starting material unless otherwise indicated).

TABLE 9

| | | Comparative |
|---|---|---|
| Ingredients (INCI name, chemical name) | Composition 9 | composition F |
| Hydrogenated Coco-Glycerides | 27 | 27 |
| (Softisan 100 - Cremer Oleo) | | |
| Ethylcellulose | 40 | 40 |
| (aqueous dispersion; 26.2% of ethylcellulose; | | |
| Aquacoat ECD-30 - FMC Biopolymer) | | |
| Octyldodecanol | 4 | 4 |
| Gellan Gum | 0.15 | 0.15 |
| (Kelcogel CG LA- CP Kelco) | | |
| Euphorbia Cerifera (Candelilla) Wax Extract | 2 | — |
| (Candellila Resin E-1 - Japan Natural Products) | | |
| Trimethyl siloxysilicate | — | 2.66 (2% |
| (as a mixture in isododecane; Silsoft 74 - | | active |
| Momentive Performances Materials) | | material) |
| Neopentyl Glycol Dicaprate | 20.85 | 20.85 |
| (Estemol N-01- Nisshin Oillio) | | |
| Cetyl alcohol | 1.00 | 1.00 |
| Water | 4.90 | 4.90 |
| Red 33 | 0.10 | 0.10 |
| % Ethylcelluose and (candelilla resin or silicone | 12.48 | 12.48 |
| resin) | | |

### Procedure

The following steps are carried out:
1. The Softisan 100 and the octyldodecanol are mixed at 60° C. and then this mixture is poured into the aqueous dispersion of ethylcellulose, with stirring using the Rayneri deflocculator for 10 minutes at 1200 rpm.
2. The gellan gum is added and the stirring is continued for 30 minutes.
3. The dyes solubilized in water are then introduced and stirring is carried out for 15 minutes.
4. Furthermore, the dicaprylyl ether and the candelilla resin are mixed and the resulting mixture is heated at 85° C. Once the mixture is homogeneous, the cetyl alcohol is added thereto with stirring. In the case of the comparative composition, trimethylsiloxysilicate is added thereto.
5. When the preparations homogeneous, it is cooled to 55° C. and is poured onto the mixture obtained at the outcome of step 3. The whole mixture is emulsified for 15 minutes and then left to cool to ambient temperature with stirring. The mixture is then packaged in a gloss bottle (pot fitted with a flocked applicator—reference 14030, Geka GmbH).

### Evaluation of the Composition

A stable, homogeneous liquid composition is obtained in each of the cases.

Each composition applies easily to the lips, as a fine and fresh deposit which is only very slightly tacky, which does not migrate and which does not transfer (protocol of Example 1).

It is noted however that the coverage and the intensity of the colour are significantly greater in the case of the composition according to the invention.

Moreover, the resistance to water and to oil are significantly greater in the case of the composition according to the invention (protocols of Example 1).

### Example 10

In the following table, the amount of each compound is given as a weight percentage/weight of composition (the percentages are expressed as weight of starting material unless otherwise indicated).

TABLE 10

| | Comparative | Composition |
|---|---|---|
| Ingredients (INCI name, chemical name) | Composition G | 10 |
| Gellan Gum | 0.2 | 0.2 |
| (Kelcogel CG LA- CP Kelco) | | |
| Sucrose Laurate (Surfhope SE COSME C-1216, | 1.5 | 1.5 |
| Mitsubishi Kagaku Foods) | | |
| Water | 2.0 | 2.0 |
| Red 33 | 0.04 | 0.04 |
| Red 7 | 0.5 | 0.5 |
| Phenoxyethanol | 0.7 | 0.7 |
| Ethylcellulose | 39.0 | 36.5 |
| (aqueous dispersion with 26.2% of ethylcellulose; | | |
| Aquacoat ECD-30 - FMC Biopolymer) | | |
| Ethylcellulose (Aqualon EC N7 PHARM, | 4.8 | 4.8 |
| ASHLAND) | | |
| Yellow 6 | 0.22 | 0.22 |
| Caprylic/Capric Triglyceride | 8.0 | 7.44 |
| Octyldodecanol | 34.04 | 33.1 |
| Hydrogenated Coco-Glycerides (Softisan 100, | 7.0 | 6.5 |
| Cremer Oleo) | | |
| Ethanol | 2.0 | 2.0 |
| Euphorbia Cerifera (Candelilla) Wax Extract | — | 4.5 |
| (Candellila Resin E-1 - Japan Natural Products) | | |

### Procedure

On the one hand, the capric/caprylic triglyceride, the Softisan 100 and part of the octyldodecanol are mixed at 55° C. with stirring, then this mixture is added to the Aquacoat ECD at 55° C. with stirring.

The gellan gum is added and stirring is continued for 30 minutes.

Finally, the sucrose laurate is added and stirring is maintained for 45 minutes.

On the other hand, the remaining octyldodecanol is mixed at 55° C. with the N7 Ethylcellulose, with stirring until a gel is obtained. In the case where the composition comprises candelilla resin, the remainder of the octyldodecanol and the N7 Ethylcellulose are mixed, with stirring at 90° C., then the candelilla resin is introduced, still with stirring, until an homogeneous product is obtained. This mixture is cooled to 60° C.

The resulting gel is added to the mixture obtained above, with stirring, for 30 minutes at 55° C. (1300 rpm).

The dyestuffs are then introduced, with stirring, at 45° C., then the preservatives. The mixture is then packaged in a gloss bottle (pot fitted with a flocked applicator—reference 14030, Geka GmbH).

### Evaluation of the Compositions

In each case, a homogeneous, stable liquid composition is obtained.

Each composition is easily applied to the lips, in a fine and fresh deposit, which does not migrate and which is only very slightly tacky.

The compositions do not transfer.

It is further noted that the resistance to water and oil of Composition 10 according to the invention are significantly improved compared to Comparative Composition G.

### Example 11

In the following table, the amount of each compound is given as a weight percentage/weight of composition (the percentages are expressed as weight of starting material unless otherwise indicated).

TABLE 11

| Ingredients (INCI name, chemical name) | Composition 11 |
|---|---|
| Ethylcellulose | 40 |
| (aqueous dispersion with 26.2% of ethylcellulose; | |
| Aquacoat ECD-30 - FMC Biopolymer) | |
| Octyldodecanol | 31 |
| Gellan gum | 0.15 |
| (Kelcogel CG LA- CP Kelco) | |
| Euphorbia Cerifera (Candelilla) Wax Extract | 2 |
| (Candellila Resin E-1 - Japan Natural Products) | |
| Neopentyl Glycol Dicaprate | 20.85 |
| (Estemol N-01- Nisshin Oillio) | |
| Cetyl alcohol | 1.00 |
| Water | 4.90 |
| Red 33 | 0.10 |

### Procedure

The neopentylglycol dicaprate, the octyldodecanol, the cetyl alcohol, the candelilla resin are mixed at 80° C. with stirring, then this mixture is cooled to 55° C. and it is added to the Aquacoat ECD at 55° C., under stirring.

The gellan gum is added and the stirring is continued for 30 minutes.

The dyestuffs are then introduced, with stirring. This mixture is cooled to 30° C. and then the preservatives are added.

The mixture is left to cool and packaged in a gloss-type bottle (bottle fitted with a flocked applicator—reference 14030, Geka GmbH).

### Evaluation of the Composition

A homogeneous, stable liquid composition is obtained.

It is easily applied to the lips, in a fine and fresh deposit, which is only very slightly tacky. The deposit does not migrate.

It is also observed that the deposit does not transfer and that the resistances to water and to oil of the composition is very good (protocols of example 1).

### Example 12

In the following table, the amount of each compound is given as a weight percentage/weight of composition (the percentages are expressed as weight of starting material unless otherwise indicated).

TABLE 12

| Ingredients (INCI name, chemical name) | Composition 12 |
|---|---|
| Octyldodecanol | 11.55 |
| Sucrose Laurate (Surfhope SE COSME C-1216 - | 1.5 |
| Mitsubishi Kagaku Foods) | |
| Phenoxyethanol | 0.7 |
| Ethylcellulose (AQUALON EC N7 PHARM - | 3 |
| Ashland) | |
| Octyldodecanol | 25.71 |
| Isononyl Isononanoate | 4.8 |
| Ethanol | 2 |
| Red 33 | 0.04 |
| Water | 7.5 |
| Caprylic/Capric Triglyceride | 8 |
| Hydrogenated Coco-Glycerides (Softisan 100 - | 7 |
| Cremer Oleo) | |
| Red 7 | 0.5 |
| Dehydroxanthan Gum | 0.2 |
| (Amaze XT - Akzo Nobel) | |
| Euphorbia Cerifera (Candelilla) Wax Extract | 3 |
| (Candellila Resin E-1 - Japan Natural Products) | |
| Ethylcellulose | 24.5 |
| (aqueous dispersion with 26.2% of ethylcellulose; | |
| Aquacoat ECD-30 - FMC Biopolymer) | |

### Procedure

On the one hand, the capric/caprylic triglyceride, the Softisan 100 and part of the octyldodecanol are mixed at 55° C. with stirring, then this mixture is added to the Aquacoat ECD at 55° C. with stirring.

The xanthan gum is added and stirring is continued for 30 minutes.

Finally, the sucrose laurate is added and stirring is maintained for 45 minutes.

The rest of the octyldodecanol and the N7 Ethylcellulose are mixed, with stirring at 90° C., then the candelilla resin is introduced, still with stirring, until a homogeneous mixture is obtained. This mixture is cooled to 60° C.

The resulting gel is added to the mixture obtained above, with stirring, for 30 minutes at 55° C. (1300 rpm).

The dyestuffs are then introduced, with stirring, at 45° C., then the preservatives.

The mixture is left to cool and packaged in a gloss-type bottle (bottle fitted with a flocked applicator—reference 14030, Geka GmbH).

### Evaluation of the Composition

A homogeneous, stable liquid composition is obtained.

It is easily applied to the lips, in a fine and fresh deposit, which is only very slightly tacky. The deposit does not migrate.

It is also observed that the deposit does not transfer and that the resistance to water and to oil of the composition is very good (protocols of example 1).

1. Liquid cosmetic composition in the form of an emulsion comprising:
from 4% to 25% by weight, relative to the total weight of the composition, expressed as active material, of ethylcellulose;
at least one natural resin;
at least one first non-volatile oil that is liquid at 25° C., chosen from saturated or unsaturated, linear or branched C10-C26 fatty alcohols;
optionally at least one of (a) at least one hydrocarbon-based compound with an ester function, which is solid at ambient temperature; (b) at least one second non-volatile polar hydrocarbon oil, liquid at ambient temperature, different from the first oil and chosen from ester, ether or carbonate oils; or (c) mixtures thereof.

2. Composition according to claim 1, characterized in that the content of ethylcellulose, expressed as active material, is from 5% to 20% by weight, relative to the total weight of the composition.

3. Composition according to claim 1, in which said at least one natural resin is chosen from: a) acaroid resins, b) ambers, c) asphaltite and gilsonite, d) Peru balsam, e) Tolu balsam, f) benzoin resins, g) Canada balsam, h) copal resins, i) damars, j) elemis, k) frankincenses, l) galbanums, m) labdanums, n) mastics, o) myrrh, p) sandarac, q) shellacs, r) styrax, s) Venice turpentine, t) rosins, tall oils, v) resins extracted from plant waxes, and mixtures of these resins.

4. Composition according to claim 1, characterized in that it comprises at least one natural resin, the INCI name of which comprises at least one of the following: Euphorbia Cerifera Wax Extract, Candellila Wax Extract, Protium Heptaphyllum Resin, Shorea Robusta Resin; and mixtures thereof.

5. Composition according to claim 1, characterized in that said natural resin contains at least 30% of terpenic compounds by weight relative to the total weight of resin.

6. Composition according to claim 1, characterized in that said natural resin comprises at least 10% by weight, of polyterpenic compounds relative to the total weight of natural resin.

7. Characterized in that said natural resin comprises less than 70% by weight of monoterpenic or sesquiterpenic compounds relative to the total weight of the natural resin.

8. Composition according to claim 1, characterized in that the natural resin has a number-average molecular weight of less than or equal to 10 000 g/mol.

9. Composition according to claim 1, characterized in that said natural resin(s) have a glass transition temperature from 0° C. to 200° C.

10. Composition according to claim 1, characterized in that the natural resin has a softening point from 20° C. to 150° C.

11. Composition according to claim 1, characterized in that the content of natural resin represents from 0.5% to 15% by weight, relative to the total weight of the composition.

12. Composition according to claim 1, characterized in that the first oil is chosen from lauryl alcohol, isostearyl alcohol, oleyl alcohol, 2-butyloctanol, 2-undecylpentadecanol, 2-hexyldecyl alcohol, isocetyl alcohol, octyldodecanol and mixtures thereof.

13. Composition according to claim 1, characterized in that the content of first oil(s) is from 0.5% to less than 20% by weight relative to the total weight of the composition.

14. Composition according to claim 1, characterized in that the content of first oil(s) is from 20 to 60% by relative to the total weight of the composition.

15. Composition according to claim 1, characterized in that the at least one hydrocarbon-based second oil that is liquid at ambient temperature is chosen from:
ethers of formula ROR′ or carbonates of formula RO(CO)OR′, in which formulae, identical or not, the R and R′ groups represent a saturated or unsaturated, branched or unbranched, hydrocarbon-based group comprising at most 16 carbon atoms;
hydroxylated or non-hydroxylated plant oils;
optionally hydroxylated ester oils comprising from 1 to 4 ester functions, of which at least one of them, which is linear or branched, saturated, unsaturated or aromatic, comprises at least 10 carbon atoms;
liquid polyesters derived from the reaction of a monounsaturated or polyunsaturated acid dimer; the fatty acid comprising from 16 to 22 carbon atoms;
and mixtures thereof.

16. Composition according to claim 1, characterized in that the content of second oil, when present, is from 2% to 35% by weight relative to the total weight of the composition.

17. Composition according to claim 1, characterized in that the hydrocarbon-based compound with an ester function, which is solid at ambient temperature, is chosen from:
plant butters,
totally or partially hydrogenated plant oils,
plant waxes,
beeswax, synthetic beeswax, lanolin wax,
esters of hydrogenated castor oil and of C16-C22 fatty acids,
saturated or unsaturated, linear or branched, optionally monohydroxylated or polyhydroxylated, optionally hydrogenated,
waxes obtained by hydrogenation of plant oil which are esterified with fatty alcohols,
esters of formula R1COOR2 in which R1 and R2 represent linear, branched or cyclic aliphatic chains in which the number of atoms ranges from 10 to 50, which may contain a heteroatom,
natural or synthetic polyalkylenated or polyglycerolated hydrocarbon-based waxes, of animal or plant origin;
polyesters derived from the condensation of a linear or branched C6-C10 dicarboxylic acid and of an ester of diglycerol and of optionally hydroxylated, linear or branched C6-C20 monocarboxylic acids,
polyesters obtained from an acid dimer, said acid being unsaturated and comprising 16 to 24 carbon atoms, and from at least one alcohol or polyol, and
mixtures thereof.

18. Composition according to claim 1, characterized in that the hydrocarbon-based compound which is solid at ambient temperature is chosen from saturated or unsaturated, linear or branched, optionally monohydroxylated or polyhydroxylated, optionally hydrogenated, fatty acid triglycerides.

19. Composition according to claim 1, characterized in that the content of hydrocarbon-based compound(s) that are solid at ambient temperature, when present, is at least 15% by weight relative to the total weight of the composition.

20. Composition according to claim 1, characterized in that the content of solid hydrocarbon compound(s) at ambient temperature, when present, is less than 15% by weight relative to the total weight of the composition.

21. Composition according to claim 1, characterized in that the composition comprises water and the water content ranges from 20% to 60% by weight relative to the total weight of the composition.

22. Composition according to claim 1, characterized in that the composition further comprises at least one C1-C5 monoalcohol.

23. Composition according to claim 1, characterized in that the composition further comprises at least one polyol that is liquid at 15° C. and atmospheric pressure.

24. Composition according to claim 1, characterized in that if the composition further comprises at least one non-volatile silicone oil, the composition has a content of non-volatile silicone oil(s) of less than or equal to 10% by weight relative to the total weight of the composition.

25. Composition according to claim 1, characterized in that if the composition further comprises at least one volatile hydrocarbon-based or silicone oil(s), the composition has a content of volatile hydrocarbon-based or silicone oil(s) of less than or equal to 10% by weight relative to the total weight of the composition.

26. Composition according to claim 1, characterized in that if the composition further comprises at least one non-polar non-volatile hydrocarbon-based oil(s), the composition has a content of non-polar non-volatile hydrocarbon-based oil(s) of less than 10% by weight, relative to the total weight of the composition.

27. Composition according to claim 1, characterized in that it further comprises at least one colorant.

28. Composition according to claim 1, characterized in that it is in the form of an oil-in-water emulsion.

29. Process for making up and/or caring for human keratin materials, comprising applying the composition of claim 1 to the keratin materials.
